SEQUENCE LISTING (30 sequences)

SEQ ID NO: 1 - protein, 8 aa, Artificial Sequence
  complementarity determining region (CDR), CDR1
GIDLSTKS

SEQ ID NO: 2 - protein, 8 aa, Artificial Sequence
  CDR2
HSSSGGTT

SEQ ID NO: 3 - protein, 6 aa, Artificial Sequence
  CDR3
TAPVGY

SEQ ID NO: 4 - protein, 8 aa, Artificial Sequence
  CDR1
GIILSSKA

SEQ ID NO: 5 - protein, 8 aa, Artificial Sequence
  CDR2
HSSVSGTT

SEQ ID NO: 6 - protein, 6 aa, Artificial Sequence
  CDR3
TAPVGH

SEQ ID NO: 7 - protein, 8 aa, Artificial Sequence
  CDR1
GITFSTKP

SEQ ID NO: 8 - protein, 8 aa, Artificial Sequence
  CDR2
HISSDGTT

SEQ ID NO: 9 - protein, 6 aa, Artificial Sequence
  CDR3
TAPVGY

SEQ ID NO: 10 - protein, 8 aa, Artificial Sequence
  CDR1
GRSFSIYG

SEQ ID NO: 11 - protein, 8 aa, Artificial Sequence
  CDR2
ITGSGLTT

SEQ ID NO: 12 - protein, 19 aa, Artificial Sequence
  CDR3
AATRAYSGYYGENPTRYAY

SEQ ID NO: 13 - protein, 111 aa, Artificial Sequence
  domain antibody (dAb) sequence
QVQLQESGGGLVQAGGSLRLSCAASGIDLSTKSVAWYRQPPGKQREGVAHSSSGGTTIYADSVKGRFTISRDNSKNTVYLEMNSLKPEDTAVYYCTAPVGYWGQGTQVTVS

SEQ ID NO: 14 - protein, 111 aa, Artificial Sequence
  dAb sequence
QVQLQESGGGLVQPGGSLRLSCAASGIILSSKAVAWYRQPPGQQREGVAHSSVSGTTIYADSVKGRFTVSRDNAKNTVYLEMNSLKPEDTAVYYCTAPVGHWGQGTQVTVS

SEQ ID NO: 15 - protein, 111 aa, Artificial Sequence
  dAb sequence
QVQLQQSGGGLVQAGGSLRLSCAASGITFSTKPVAWYRQPPGKQREGVAHISSDGTTIYADSVKGRFTVSRDNAKNTVYLEMNSLKPEDTGVYYCTAPVGYWGQGTQVTVS

SEQ ID NO: 16 - protein, 125 aa, Artificial Sequence
  dAb sequence
QVQLQQSGGGLVQAGGSLRLACAASGRSFSIYGITWFRQAPGKEREFVAAITGSGLTTNYADSVKGRFTISRDNAKNMVYLQLNSLKPDDTAVYYCAATRAYSGYYGENPTRYAYWGQGTQVTVS

SEQ ID NO: 17 - protein, 502 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQSQQEGIKM

SEQ ID NO: 18 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 19 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 20 - protein, 414 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 21 - protein, 20 aa, Homo sapiens
MHYCVLSAFLILHLVTVALS

SEQ ID NO: 22 - protein, 282 aa, Homo sapiens
LSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKR

SEQ ID NO: 23 - protein, 112 aa, Homo sapiens
ALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 24 - protein, 412 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 25 - protein, 394 aa, Artificial Sequence
  sequence of a TGFbeta monomer (signal peptide and latency associated peptide)
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFRKDLGWKWIHEPKGYNANFCAGACPYRASKSPSCVSQDLEPLTIVYYVGRKPKVEQLSNMIVKSCKCS

SEQ ID NO: 26 - protein, 112 aa, Artificial Sequence
  signal peptide sequence
MHYCVLSAFLILHLVTVALSALDAAYCFRNVQDNCCLRPLYIDFRKDLGWKWIHEPKGYNANFCAGACPYRASKSPSCVSQDLEPLTIVYYVGRKPKVEQLSNMIVKSCKCS

SEQ ID NO: 27 - DNA, 30 bp, Artificial Sequence
  NFAT response element
ggaggaaaaactgtttcatacagaaggcgt

SEQ ID NO: 28 - protein, 4 aa, Artificial Sequence
  ITAM (immunoreceptor tyrosine-based activation motif)
  misc_feature (2)..(3): Xaa can be any naturally occurring amino acid
  MISC_FEATURE (4)..(4): Xaa may be Leu or Ile
YXXX

SEQ ID NO: 29 - protein, 4 aa, Artificial Sequence
  basic amino acid furin target sequence
  misc_feature (2)..(2): Xaa can be any naturally occurring amino acid
  MISC_FEATURE (3)..(3): Xaa may be Arg or Lys
RXXR

SEQ ID NO: 30 - protein, 7 aa, Artificial Sequence
  consensus Tobacco Etch Virus (TEV) cleavage site
ENLYFQS

(19) United States
(12) Patent Application Publication
(10) Pub. No.: US 2021/0244762 A1
(43) Pub. Date: Aug. 12, 2021

(54) CAR-T CELLS EXPRESSING AN INHIBITORY ANTI-TGF-BETA-RECEPTOR II SINGLE DOMAIN ANTIBODY

(71) Applicant: AUTOLUS LIMITED, London (GB)

(72) Inventors: Martin Pulé, London (GB); Shaun Cordoba, London (GB); Shimobi Onuoha, London (GB); Alex Kinna, London (GB); Simon Thomas, London (GB); Mathieu Ferrari, London (GB); Marco Della Peruta, London (GB); Phillip Wu, London (GB)

(21) Appl. No.: 17/053,597

(22) PCT Filed: May 10, 2019
(86) PCT No.: PCT/GB2019/051284

§ 371 Date: Nov. 6, 2020

(30) Foreign Application Priority Data
May 11, 2018 (GB) 1807693.5

(51) Int. Cl. A61K 35/17; C07K 16/28; C07K 14/725

(52) U.S. Cl. CPC A61K 35/17; C07K 16/2863; C07K 2317/569; C07K 2317/565; C07K 14/7051

(57) ABSTRACT

The present invention relates to a cell which comprises a chimeric antigen receptor (CAR) or transgenic T cell receptor (TCR) and secretes an antibody which binds a transforming growth factor beta receptor (TβR).

### FIELD OF THE INVENTION

The present invention relates to a cell which comprises a chimeric antigen receptor (CAR) or transgenic T cell receptor (TCR) and secretes a factor which binds a transforming growth factor beta receptor (TβR). The factor may be an antibody.

### BACKGROUND TO THE INVENTION

Adoptive immunotherapy of cancer involves the ex vivo generation of cancer-antigen specific cells and their administration. Adoptively transferred immune effector cells also activate existing adaptive and innate immune cells within the tumour once they activate and start causing inflammation.

The native specificity of immune effector cells can be exploited in adoptive immunotherapy—for example during the generation of melanoma specific T-cells from expansion of tumour infiltrating lymphocytes in tumour resections. Otherwise a specificity can be grafted onto a T-cell using genetic engineering. Two common methods for achieving this are using chimeric antigen receptors or transgenic T-cell receptors. Different kinds of immune effector cells can also be used. For example, alpha/beta T-cells, NK cells, gamma delta T-cells or macrophages can be used.

Adoptive immunotherapy has been successful in treating a number of lymphoid malignancies, such as B-cell Acute Lymphoblastic Leukaemia (B-ALL), Diffuse Large B-cell Lymphoma (DLBCL) and Multiple Myeloma (MM), however there has been relatively little success in the treatment of other cancers.

Engineered cells face hostile microenvironments which limit adoptive immunotherapy. Modulating the tumour microenvironment may convert the microenvironment into a more favourable environment which enables the engineered immune effector cells to proliferate, survive and/or engraft thereby providing a more effective engineered cell therapy. One of the main inhibitory mechanisms within the tumour microenvironment is transforming growth factor beta (TGFβ).

The use of systemic therapeutic agents which block TGFβ has been tested. For example Fresolimumab is a neutralizing antibody which blocks TGFβ1-3. Fresolimumab has been tested in metastatic melanoma and high-grade glioma. This showed some effectiveness in the enhancement of a tumour-specific immune response but failed to eradicate the tumour.

Other approaches include small molecules which inhibit SMAD signalling, downstream of transforming growth factor beta receptor (TβR). The best characterized example is Galunisertib which has been tested as a monotherapy or in combination with alkylating agents, Lomustine or temozolamide for glioblastoma and other combinations. These approaches have focused on the inhibitory microenvironment and have not been particularly effective.

Accordingly, there remains a need for approaches to produce immune effector cells which are capable of tolerating the tumour microenvironment e.g. which are less susceptible to TGFβ, which may improve the effectiveness of engineered immune effector cells to proliferate, survive and/or engraft in the microenvironment.

### SUMMARY OF THE INVENTION

The present inventors have developed a cell with an in-built system to control TGFβ signalling, rendering the cell less susceptible (i.e. more resistant) to TGFβ.

Accordingly, in a first aspect, the present invention provides a cell which comprises a chimeric antigen receptor (CAR) or transgenic T cell receptor (TCR) and secretes a factor which binds a transforming growth factor beta receptor (TβR). The secreted factor may block the interaction between TβR and TGFβ.

The secreted factor may bind TGF-beta receptor type-2 (TβRII).

The secreted factor may comprise a variant TGFβ polypeptide, a mutant TGFβ or a truncated TGFβ polypeptide.

The secreted factor may be or comprise an antibody.

The antibody may be a domain antibody (dAb), for example a dAb which comprises one of the following sets of complementarity determining regions (CDRs):

a)
CDR1
(SEQ ID No. 1)
GIDLSTKS;
CDR2
(SEQ ID No. 2)
HSSSGGTT;
and
CDR3
(SEQ ID No. 3)
TAPVGY;
b)
CDR1
(SEQ ID No. 4)
GIILSSKA;
CDR2
(SEQ ID No. 5)
HSSVSGTT;
and
CDR3
(SEQ ID No. 6)
TAPVGH;
c)
CDR1
(SEQ ID No. 7)
GITFSTKP;
CDR2
(SEQ ID No. 8)
HISSDGTT;
and
CDR3
(SEQ ID No. 9)
TAPVGY;
or
d)
CDR1
(SEQ ID No. 10)
GRSFSIYG;
CDR2
(SEQ ID No. 11)
ITGSGLTT;
and
CDR3
(SEQ ID No. 12)
AATRAYSGYYGENPTRYAY.

The cell may secrete a dAb comprising one of the sequences shown as SEQ ID No. 13, 14, 15 or 16.

In a second aspect, the present invention provides a domain antibody (dAb) which binds transforming growth factor beta receptor type II (TβRII) and comprises one of the following sets of complementarity determining regions (CDRs):

a)
CDR1
(SEQ ID No. 1)
GIDLSTKS;
CDR2
(SEQ ID No. 2)
HSSSGGTT;
and
CDR3
(SEQ ID No. 3)
TAPVGY;
b)
CDR1
(SEQ ID No. 4)
GIILSSKA;
CDR2
(SEQ ID No. 5)
HSSVSGTT;
and
CDR3
(SEQ ID No. 6)
TAPVGH;
c)
CDR1
(SEQ ID No. 7)
GITFSTKP;
CDR2
(SEQ ID No. 8)
HISSDGTT;
and
CDR3
(SEQ ID No. 9)
TAPVGY;
or
d)
CDR1
(SEQ ID No. 10)
GRSFSIYG;
CDR2
(SEQ ID No. 11)
ITGSGLTT;
and
CDR3
(SEQ ID No. 12)
AATRAYSGYYGENPTRYAY.

The dAb may comprise one of the sequences shown as SEQ ID No. 13, 14, 15 or 16.

In a third aspect, the present invention provides a nucleic acid sequence encoding a dAb according to the second aspect of the invention.

In a fourth aspect, the present invention provides a nucleic acid construct which comprises: (i) a first polynucleotide which encodes a secreted factor or antibody as defined in the first aspect of the invention; and (ii) a second polynucleotide which encodes a chimeric antigen receptor (CAR) or a transgenic T-cell receptor (TCR).

The first and second polynucleotides may be separated by a co-expression site.

In a fifth aspect, the present invention provides a kit of polynucleotides comprising:

(i) a first polynucleotide which encodes a secreted factor or antibody as defined in the first aspect of the invention; and

(ii) a second polynucleotide which encodes a chimeric antigen receptor (CAR) or a transgenic (TCR).

In a sixth aspect the present invention provides a vector which comprises a nucleic acid sequence according to the third aspect of the invention or a nucleic acid construct according to the fourth aspect of the invention.

In a seventh aspect the present invention provides a kit of vectors which comprises:

(i) a first vector which comprises a polynucleotide which encodes a secreted factor or antibody as defined in the first aspect of the invention; and

(ii) a second vector which comprises a polynucleotide which encodes a chimeric antigen receptor (CAR) or a transgenic T-cell receptor (TCR).

In an eighth aspect the present invention provides a pharmaceutical composition which comprises a cell according to the first aspect of the invention or a dAb according to the second aspect of the invention.

In a ninth aspect the present invention provides a pharmaceutical composition according to the eighth aspect of the invention, for use in treating and/or preventing a disease.

In a tenth aspect the present invention provides a method for treating and/or preventing a disease, which comprises the step of administering a pharmaceutical composition according to the eighth aspect of the invention to a subject in need thereof.

In an eleventh aspect the present invention provides the use of a cell according to the first aspect of the invention, or a dAb according to the second aspect of the invention in the manufacture of a medicament for the treatment and/or prevention of a disease.

The disease may be cancer.

In a twelfth aspect the present invention provides method for making a cell according to the first aspect of the invention, which comprises the step of introducing: a nucleic acid sequence according to third aspect of the invention, or a nucleic acid construct according to the fourth aspect of the invention, a first polynucleotide and a second polynucleotide as defined in the fifth aspect of the invention, a vector according to the sixth aspect of the invention or a first and second vector as defined in the seventh aspect of the invention into a cell ex vivo.

Further aspects of the invention are summarised in the following numbered paragraphs:

A1. A cell which comprises a chimeric antigen receptor (CAR) or transgenic T cell receptor (TCR) and secretes a domain antibody (dAb).

A2. A cell according to paragraph A1, wherein the dAb binds transforming growth factor beta (TGFβ) or a TGFβ receptor (TβR), PD-1, PD-L1, LAG-3 or CTLA-4.

A3. A nucleic acid construct which comprises: (i) a first polynucleotide which encodes a domain antibody (dAb); and (ii) a second polynucleotide which encodes a chimeric antigen receptor (CAR) or a transgenic T-cell receptor (TCR).

A4. A kit of polynucleotides comprising:

(i) a first polynucleotide which encodes a domain antibody (dAb); and

(ii) a second polynucleotide which encodes a chimeric antigen receptor (CAR) or a transgenic (TCR).

A5. A vector which comprises a nucleic acid construct according to paragraph A3.

A6. A kit of vectors which comprises:

(i) a first vector which comprises a polynucleotide which encodes a domain antibody (dAb); and

(ii) a second vector which comprises a polynucleotide which encodes a chimeric antigen receptor (CAR) or a transgenic T-cell receptor (TCR).

A7. A pharmaceutical composition which comprises a cell according to paragraph A1 or A2.

A8. A pharmaceutical composition according to paragraph A7, for use in treating and/or preventing a disease.

A9. A method for treating and/or preventing a disease, which comprises the step of administering a pharmaceutical composition according to paragraph A7 to a subject in need thereof.

A10. The use of a cell according to paragraph A1 or A2 in the manufacture of a medicament for the treatment and/or prevention of a disease.

A11. The pharmaceutical composition for use according to paragraph A8, the method according to paragraph A9, or the use according to paragraph A10, wherein the disease is cancer.

A12. A method for making a cell according to paragraph A1 or A2, which comprises the step of introducing: a nucleic acid construct according to paragraph A3, a first polynucleotide and a second polynucleotide as defined in paragraph A4, a vector according to paragraph A5 or a first and second vector as defined in paragraph A6 into a cell ex vivo.

B1. A cell which comprises a chimeric antigen receptor (CAR) or transgenic T cell receptor (TCR) and secretes a Fab antibody (Fab).

B2. A cell according to paragraph B1, wherein the Fab antibody binds transforming growth factor beta (TGFβ) or a TGFβ receptor (TβR), PD-1, PD-L1, LAG-3 or CTLA-4.

B3. A nucleic acid construct which comprises: (i) a first polynucleotide which encodes a Fab antibody (Fab); and (ii) a second polynucleotide which encodes a chimeric antigen receptor (CAR) or a transgenic T-cell receptor (TCR).

B4. A kit of polynucleotides comprising:

(i) a first polynucleotide which encodes a Fab antibody (Fab); and

(ii) a second polynucleotide which encodes a chimeric antigen receptor (CAR) or a transgenic (TCR).

B5. A vector which comprises a nucleic acid construct according to paragraph B3.

B6. A kit of vectors which comprises:

(i) a first vector which comprises a polynucleotide which encodes a Fab antibody (Fab); and

(ii) a second vector which comprises a polynucleotide which encodes a chimeric antigen receptor (CAR) or a transgenic T-cell receptor (TCR).

B7. A pharmaceutical composition which comprises a cell according to paragraph B1 or B2.

B8. A pharmaceutical composition according to paragraph B7, for use in treating and/or preventing a disease.

B9. A method for treating and/or preventing a disease, which comprises the step of administering a pharmaceutical composition according to paragraph B7 to a subject in need thereof.

B10. The use of a cell according to paragraph B1 or B2 in the manufacture of a medicament for the treatment and/or prevention of a disease.

B11. The pharmaceutical composition for use according to paragraph B8, the method according to paragraph B9, or the use according to paragraph B10, wherein the disease is cancer.

B12. A method for making a cell according to paragraph B1 or B2, which comprises the step of introducing: a nucleic acid construct according to paragraph B3, a first polynucleotide and a second polynucleotide as defined in paragraph B4, a vector according to paragraph B5 or a first and second vector as defined in paragraph B6 into a cell ex vivo.

The present invention provides cells which secrete a secreted factor or antibody which binds a transforming growth factor beta receptor (TβR). In this respect, the cells of the present invention are capable of modulating the inhibitory microenvironment and preventing inhibition of immune effector cells, thereby augmenting the ability of CAR- or TCR-expressing cells to attack the tumour.

The cells of the present invention will also support other cells which are known to be affected by TGFβ, including B-cells (IgA class switching and promotes decrease in activation and apoptosis), NK cells (decreased cytotoxicity and decreased chemotaxis), neutrophils (decreased effector function and promotes N1 to N2 differentiation), macrophages (decrease effector function, decreased antigen presentation and increased inflammatory cytokine secretion as well as promoting M2 differentiation over M1) and finally dendritic cells (decreased maturation, decreased antigen presentation and decreased chemotaxis).

Furthermore, in addition to supporting immune cells, the cells of the present invention are also able to modulate the activity other cells within the tumour which respond to TGFβ, such as stromal cells which are involved in fibrosis and the survival of cancer stem cells.

### BRIEF DESCRIPTION OF THE FIGURES

FIG. 1—Schematic showing different generations of chimeric antigen receptors. The basic architecture of a canonical CAR is shown as well as different iterations of the three generations of this form of receptor.

FIG. 2—Schematic showing a CAR T-cell with enhanced with TGFβ resistance according to the present invention. A CAR T-cell secretes a factor which binds to part of the receptor complex of TGFβ. As a result, inhibitory effects on the CAR T-cell itself are blocked. Further, inhibitory effects on surrounding cells are also blocked.

FIG. 3—Schematic showing the mechanism of TGFβ signalling. Panel (a) shows a receptive cell expressing both type I and II receptors which are in solution in the cell membrane. They are not associated. Panel (b) shows a secreted activated TGFβ which forms a homodimer which binds two type I and two type II receptors. This complex results in the approximation of the two receptors which then results in their phosphorylation and downstream signalling

FIG. 4—Schematic showing an embodiment of the present invention comprising dominant negative TGFβ. Panel (a) shows normal activation by TGFβ. Panel (b) shows a dominant negative TGFβ which has been generated by mutating binding sites for one of the receptor types (shown with black arrows). Binding does not result in signalling since association between type I and II receptors does not occur. Binding of wild-type TGFβ is prevented.

FIG. 5—Schematic showing an embodiment of the present invention comprising a truncated dominant negative TGFβ. Panel (a) shows normal activation by TGF. Panel (b) shows a variant TGFβ which comprises mutations and deletions that prevent homodimerization and prevent signalling.

FIG. 6—Schematic showing an embodiment of the present invention comprising blocking of TGFβ by scFv (panel (b)). Panel (a) shows normal activation by TGF.

FIG. 7—Schematic showing an embodiment of the present invention comprising blocking of TGFβ by a dAb (panel (b)). Panel (a) shows normal activation by TGF.

FIG. 8—Anti-TGFbRII dAb titration (TGFbRII-Fc)

FIG. 9—TGFb-dAb competition ELISA (0.25 μg/ml)

FIG. 10—Biacore competition ELISA

A. A schematic diagram illustrating the incremental response units (RU) of the sensograms. First RU increase (red) represents anti-TGFbRII VHH-Fc binding to the chip surface. Second RU increase (green) represents soluble TGFbRII binding to the captured antibody. The third RU increase (blue) represents the binding of TGFbeta 1 to the captured receptor.

B. The results obtained for dAb E11. Blue line=TGFb 0 mM; Yellow line=TGFb 300 nM. Sensograms for the TGFbeta 0 and 300 nM injections are superimposed to facilitate identification of TGFbeta 1 binding event (none detected).

FIG. 11—Flow cytometry assay showing binding of the anti-TGFbRII dAbs (C6, G5, E11 and H3) to cells expressing dominant negative TGFbRII.

FIG. 12—Cytotoxicity assay showing the % survival of target cells following 5 days co-culture with CAR-expressing cells which either secrete no antibody or secrete one of the anti-TGFbRII dAbs (C6, G5, E11 or H3) in the presence or absence of soluble TGFβ1.

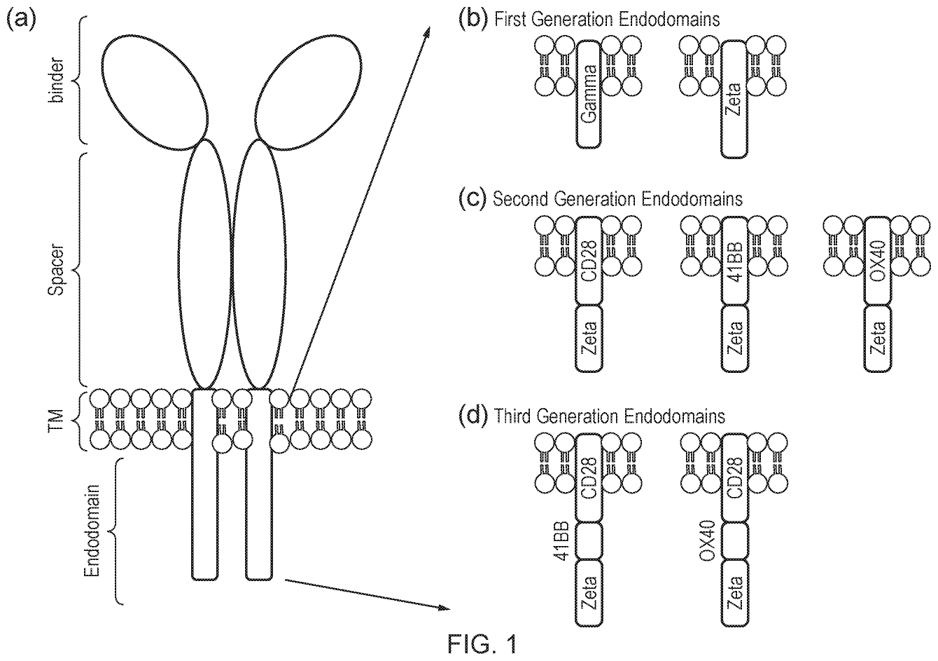

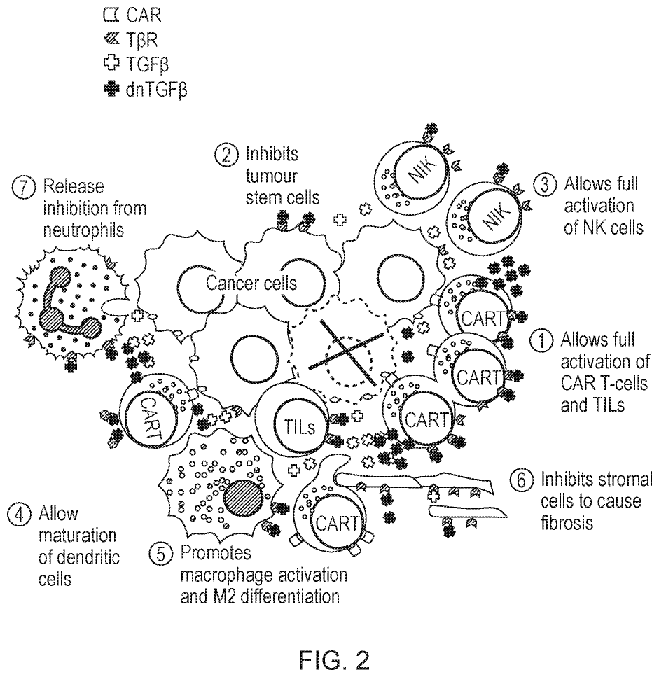

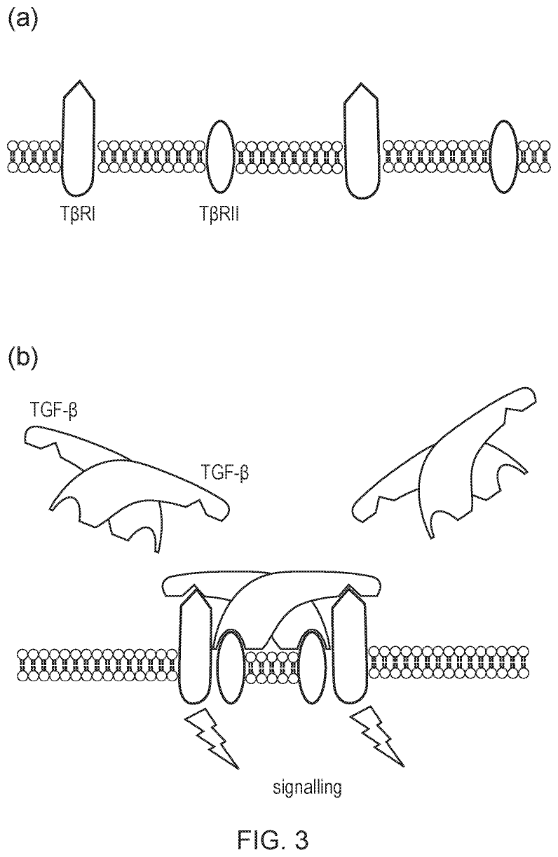

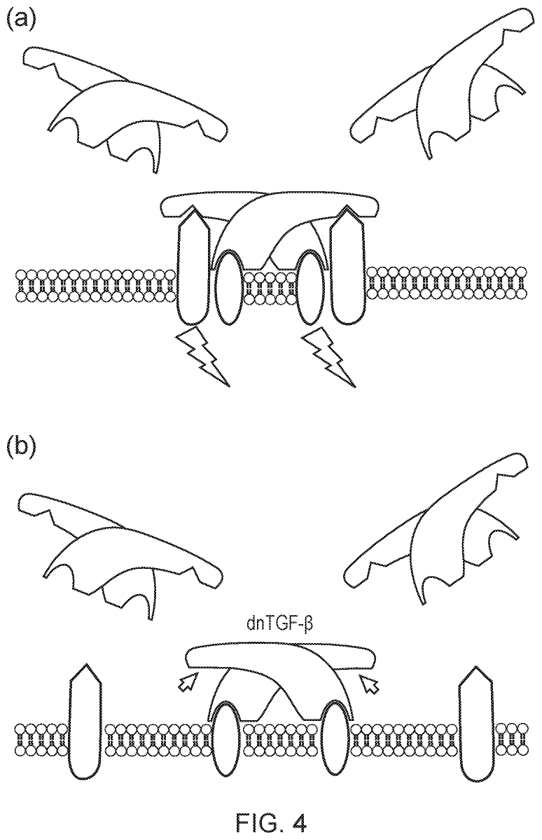

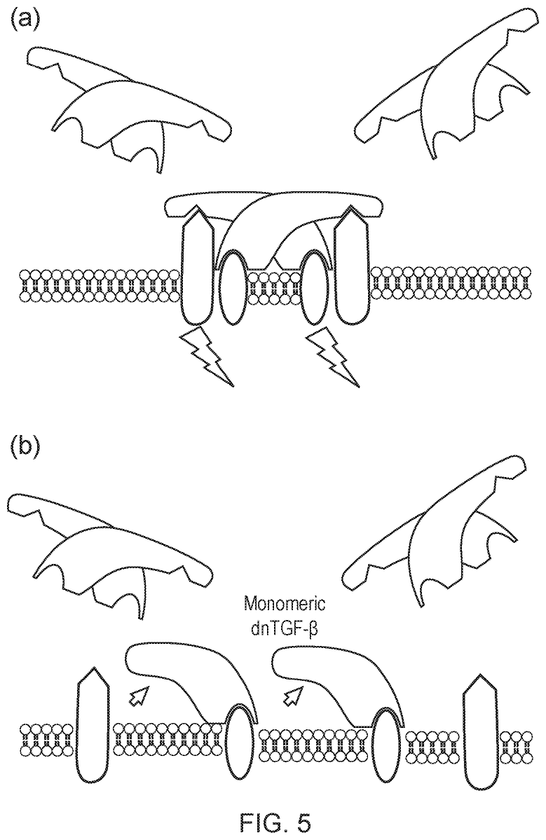

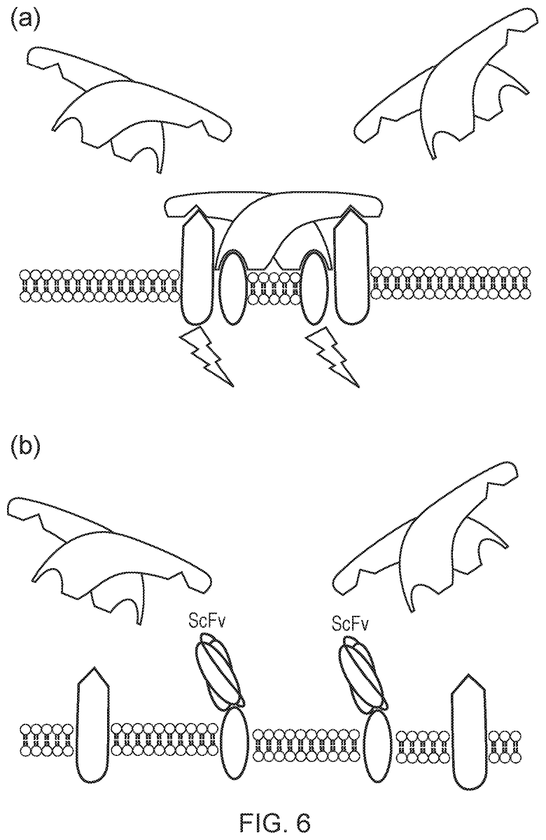

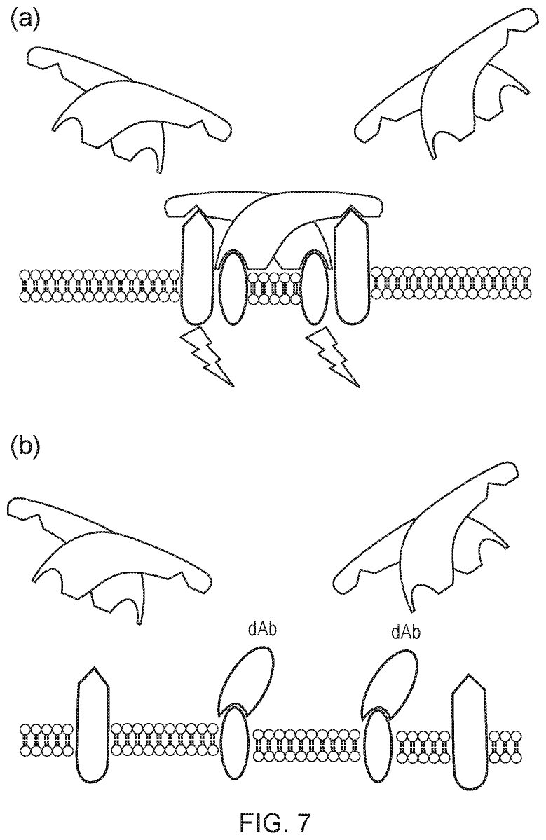

### DETAILED DESCRIPTION OF THE INVENTION

### TGFβ Biology

Transforming growth factor beta (TGF-β) is a cytokine belonging to the transforming growth factor superfamily.

The transforming growth factor beta receptors are a superfamily of serine/threonine kinase receptors. These receptors bind members of the TGFβ superfamily of growth factor and cytokine signalling proteins. There are five type II receptors (which are activatory receptors) and seven type I receptors (which are signalling propagating receptors). Type I receptors are also known as activin receptor-like kinases (ALKS).

Auxiliary co-receptors (also known as type III receptors) also exist. Each subfamily of the TGFβ superfamily of ligands binds to type I and type II receptors.

The amino acid sequence of human TGF-beta receptor type-1 (TβRI) is available from UniProt accession P36897 is shown below as SEQ ID NO: 17.

(SEQ ID NO: 17)
MEAAVAAPRPRLLLLVLAAAAAAAAALLPGATALQCFCHLCTKDNFTCVT
DGLCFVSVTETTDKVIHNSMCIAEIDLIPRDRPFVCAPSSKTGSVTTTYC
CNQDHCNKIELPTTVKSSPGLGPVELAAVIAGPVCFVCISLMLMVYICHN
RTVIHHRVPNEEDPSLDRPFISEGTTLKDLIYDMTTSGSGSGLPLLVQRT
IARTIVLQESIGKGRFGEVWRGKWRGEEVAVKIFSSREERSWFREAEIYQ
TVMLRHENILGFIAADNKDNGTWTQLWLVSDYHEHGSLFDYLNRYTVTVE
GMIKLALSTASGLAHLHMEIVGTQGKPAIAHRDLKSKNILVKKNGTCCIA
DLGLAVRHDSATDTIDIAPNHRVGTKRYMAPEVLDDSINMKHFESFKRAD
IYAMGLVFWEIARRCSIGGIHEDYQLPYYDLVPSDPSVEEMRKVVCEQKL
RPNIPNRWQSCEALRVMAKIMRECWYANGAARLTALRIKKTLSQSQQEGI
KM.

The amino acid sequence of human TGF-beta receptor type-2 (TβRII) is available from UniProt accession P37173 is shown below as SEQ ID NO: 18.

(SEQ ID NO: 18)
MGRGLLRGLW PLHIVLWTRI ASTIPPHVQK SVNNDMIVTD
NNGAVKFPQLCKFCDVRFST CDNQKSCMSN CSITSICEKP
QEVCVAVWRK NDENITLETV CHDPKLPYHD FILEDAASPK
CIMKEKKKPG ETFFMCSCSS DECNDNIIFS EEYNTSNPDL
LLVIFQVTGI SLLPPLGVAI SVIIIFYCYR VNRQQKLSST
WETGKTRKLM EFSEHCAIIL EDDRSDISST CANNINHNTE
LLPIELDTLV GKGRFAEVYK AKLKQNTSEQ FETVAVKIFP
YEEYASWKTE KDIFSDINLK HENILQFLTA EERKTELGKQ
YWLITAFHAK GNLQEYLTRH VISWEDLRKLGSSLARGIAH
LHSDHTPCGRPKMPIVHRDLKSSNILVKNDLTCCLCDFGLSLRLD
PTLSVDDLANSGQVGTARYMAPEVLESRMNLENVESFKQTDVYSM
ALVLWEMTSRCNAVGEVKDYEPPFGSKVREHPCVESMKDNVLRDR
GRPEIPSFWLNHQGIQMVCETLTECWDHDPEARLTAQCVAERFSE
LEHLDRLSGR SCSEEKIPED GSLNTTK.

Variant sequences of SEQ ID NO: 17 and 18 may have at least 80%, 85%, 90%, 95%, 98% or 99% sequence identity to SEQ ID NO: 17 and 18 respectively. Suitably, the variant sequence of SEQ ID NO: 17 or 18 is able to function as TGFβ receptor.

The three transforming growth factors have many activities. TGFβ1 and 2 are implicated in cancer, where they may stimulate the cancer stem cell, increase fibrosis/desmoplastic reactions and suppress immune recognition of the tumour.

TGFβ1, 2 and 3 signal via binding to receptors TβRII and then association to TβRI and in the case of TGFβ2 also to TβRIII. This leads to subsequent signalling through SMADs via TβRI.

TGFβs are typically secreted in the pre-pro-form. The “pre” is the N-terminal signal peptide which is cleaved off upon entry into the endoplasmic reticulum (ER). The “pro” is cleaved in the ER but remains covalently linked and forms a cage around the TGFβ called the Latency Associated Peptide (LAP). The cage opens in response to various proteases including thrombin and metalloproteases amongst others. The C-terminal region becomes the mature TGFβ molecule following its release from the pro-region by proteolytic cleavage. The mature TGFβ protein dimerizes to produce an active homodimer.

The TGFβ homodimer interacts with a LAP derived form the N-terminal region of the TGFβ gene product, forming a complex called Small Latent Complex (SLC). This complex remains in the cell until it is bound by another protein, an extracellular matrix (ECM) protein called Latent TGFβ binding protein (LTBP) which together forms a complex called the large latent complex (LLC). LLC is secreted to the ECM. TGFβ is released from this complex to a biologically active form by several classes of proteases including metalloproteases and thrombin.

A variety of cancerous tumour cells are known to produce TGFβ directly. In addition to the TGFβ production by cancerous cells, TGFβ can be produced by the wide variety of non-cancerous cells present at the tumour site. Specifically, tumour-associated T cells, natural killer (NK) cells, macrophages, epithelial cells and stromal cells have all been shown to produce TGF-β in various tumour models.

### Secreted Factor

In one aspect, the present invention provides a cell comprising a polynucleotide encoding a secreted factor which is capable of binding a transforming growth factor beta receptor (TβR) and disrupting its interaction with transforming growth factor beta (TGFβ).

As used herein, “secreted factor” is a protein which is secreted by a cell. The terms protein and polypeptide are used synonymously herein.

Secretory proteins typically comprise an N-terminal signal peptide so that when the secretory protein is expressed inside a cell, the nascent protein is directed to the ER. The classical protein secretion pathway is through the endoplasmic reticulum (ER).

### Variant TGFβ.

In one embodiment, the secreted factor comprises a dominant negative TGFβ.

“Dominant negative TGFβ” or dnTGFβ as used herein means that the secreted factor TGFβ acts antagonistically to the wild-type TGFβ.

Suitably, variants of TGFβ as disclosed herein may inhibit the function of their natural or wild-type counterparts. Suitably, the variants of TGFβ may inhibit signalling induced by wild-type TGFβ and thus neutralise its biological effects.

Suitably, binding of the dnTGFβ to TβR does not induce productive signalling downstream of TβR.

In one embodiment, the secreted factor comprises a variant TGFβ polypeptide. For example a variant TGFβ encompasses a mutant TGFβ or truncated TGFβ as described herein.

As used herein, “variant TGFβ polypeptide” means the polypeptide has an amino acid sequence which has one, two, three or more additions, deletions and/or substitutions compared with the wild-type TGFβ polypeptide.

The variant TGFβ polypeptide has less than 100% sequence identity to a wild-type TGFβ polypeptide. Suitably, the variant TGFβ polypeptide may have at least 80%, at least 85%, at least 90%, at least 95%, at least 98% or 99% sequence identity to a wild-type TGFβ polypeptide. Suitably, the variant TGFβ is able to bind a TβR and disrupt its interaction with TGFβ.

The percentage identity between two polypeptide sequences may be readily determined by programs such as BLAST, which is freely available at http://blast.ncbi.nlm.nih.gov. Suitably, the percentage identity is determined across the entirety of the reference and/or the query sequence.

Suitably, the wild-type polypeptide of TGFβ may comprise a signal peptide and a latency associated pro-peptide.

Suitably, the wild-type polypeptide of TGFβ may comprise a signal peptide.

Suitably, the secreted factor may comprise a variant mature TGFβ polypeptide.

Suitably, the wild-type TGFβ polypeptide may be TGFβ1. Suitably, the wild-type TGFβ polypeptide may comprise the amino as set forth in UniProt accession P01137.

The amino acid sequence of wild-type TGFβ1 from UniProt accession P01137 is set forth in SEQ ID NO: 19. Suitably, the wild-type TGFβ may comprise the sequence set forth in SEQ ID NO: 1. SEQ ID NO: 19.

(SEQ ID NO: 19)
MPPSGLRLLLLLLPLLWLLVLTPGRPAAGLSTCKTIDMELVKRKRIEAIR
GQILSKLRLASPPSQGEVPPGPLPEAVLALYNSTRDRVAGESAEPEPEPE
ADYYAKEVTRVLMVETHNEIYDKFKQSTHSIYMFFNTSELREAVPEPVLL
SRAELRLLRLKLKVEQHVELYQKYSNNSWRYLSNRLLAPSDSPEWLSFDV
TGVVRQWLSRGGEIEGFRLSAHCSCDSRDNTLQVDINGFTTGRRGDLATI
HGMNRPFLLLMATPLERAQHLQSSRHRRALDTNYCFSSTEKNCCVRQLYI
DFRKDLGWKWIHEPKGYHANFCLGPCPYIWSLDTQYSKVLALYNQHNPGA
SAAPCCVPQALEPLPIVYYVGRKPKVEQLSNMIVRSCKCS.

Suitably, the secreted factor may comprise a variant of SEQ ID NO: 19.

Suitably, the wild-type TGFβ polypeptide may be TGFβ2. Suitably, the wild-type TGFβ polypeptide may comprise the amino as set forth in UniProt accession P61812.

The amino acid sequence of wild-type TGFβ2 from UniProt accession P61812 is set forth in SEQ ID NO: 20. Suitably, the wild-type TGFβ may comprise the sequence set forth in SEQ ID NO: 20. SEQ ID NO: 20 comprises a pre-pro-sequence.

(SEQ ID NO: 20)
MHYCVLSAFLILHLVTVALSLSTCSTLDMDQFMRKRIEAIRGQILSKLKL
TSPPEDYPEPEEVPPEVISIYNSTRDLLQEKASRRAAACERERSDEEYYA
KEVYKIDMPPFFPSENAIPPTFYRPYFRIVRFDVSAMEKNASNLVKAEFR
VFRLQNPKARVPEQRIELYQILKSKDLTSPTQRYIDSKVVKTRAEGEWLS
FDVTDAVHEWLHHKDRNLGFKISLHCPCCTFVPSNNYIIPNKSEELEARF
AGIDGTSTYTSGDQKTIKSTRKKNSGKTPHLLLMLLPSYRLESQQTNRRK
KRALDAAYCFRNVQDNCCLRPLYIDFKRDLGWKWIHEPKGYNANFCAGAC
PYLWSSDTQHSRVLSLYNTINPEASASPCCVSQDLEPLTILYYIGKTPKI
EQLSNMIVKSCKCS.

Suitably, the secreted factor may comprise a variant of SEQ ID NO: 20.

The signal peptide of wild-type TGFβ2 which is comprise in SEQ ID NO: 20 is set forth in SEQ ID NO: 21.

(SEQ ID NO: 21)
MHYCVLSAFLILHLVTVALS

Suitably, the signal peptide may comprise the amino acid sequence set forth in SEQ ID NO: 21 or a variant thereof. The variant signal peptide may have at least 80%, 85%, 90%, 95%, 98% or 99% sequence identity to SEQ ID NO: 21, provided that the sequence provides an effective signal peptide. That is, provided that the polypeptide is capable of directing a newly synthesized secreted factor to the secretory pathway.

The latency-associated peptide fragment of wild-type TGFβ2 is set forth in SEQ ID NO: 22.

(SEQ ID NO: 22)
LSTCSTLDMDQFMRKRIEAIRGQILSKLKLTSPPEDYPEPEEVPPEVISI
YNSTRDLLQEKASRRAAACERERSDEEYYAKEVYKIDMPPFFPSENAIPP
TFYRPYFRIVRFDVSAMEKNASNLVKAEFRVFRLQNPKARVPEQRIELYQ
ILKSKDLTSPTQRYIDSKVVKTRAEGEWLSFDVTDAVHEWLHHKDRNLGF
KISLHCPCCTFVPSNNYIIPNKSEELEARFAGIDGTSTYTSGDQKTIKST
RKKNSGKTPHLLLMLLPSYRLESQQTNRRKKR

Suitably, the latency-associated peptide may comprise the amino acid sequence set forth in SEQ ID NO: 22 or a variant thereof. The variant latency associated peptide may have at least 80%, 85%, 90%, 95%, 98% or 99% sequence identity to SEQ ID NO: 22, provided that the peptide is capable of interacting with TGFβ homodimer and forming a small latent complex.

The mature protein of wild-type TGFβ2 is set forth in SEQ ID NO: 23.

(SEQ ID NO: 23)
ALDAAYCFRNVQDNCCLRPLYIDFKRDLGWKWIHEPKGYNANFCAGACPY
LWSSDTQHSRVLSLYNTINPEASASPCCVSQDLEPLTILYYIGKTPKIEQ
LSNMIVKSCKCS.

Suitably, the secreted factor may comprise a variant of SEQ ID NO: 23.

Suitably, the wild-type TGFβ polypeptide may be TGFβ3. Suitably, the wild-type TGFβ polypeptide may comprise the amino as set forth in UniProt accession P10600.

The amino acid sequence of wild-type TGFβ3 from UniProt accession P10600 is set forth in SEQ ID NO: 24. Suitably, the wild-type TGFβ may comprise the sequence set forth in SEQ ID NO: 24.

(SEQ ID NO: 24)
MKMHLQRALVVLALLNFATVSLSLSTCTTLDFGHIKKKRVEAIRGQILSK
LRLTSPPEPTVMTHVPYQVLALYNSTRELLEEMHGEREEGCTQENTESEY
YAKEIHKFDMIQGLAEHNELAVCPKGITSKVFRFNVSSVEKNRTNLFRAE
FRVLRVPNPSSKRNEQRIELFQILRPDEHIAKQRYIGGKNLPTRGTAEWL
SFDVTDTVREWLLRRESNLGLEISIHCPCHTFQPNGDILENIHEVMEIKF
KGVDNEDDHGRGDLGRLKKQKDHHNPHLILMMIPPHRLDNPGQGGQRKKR
ALDTNYCFRNLEENCCVRPLYIDFRQDLGWKVVVHEPKGYYANFCSGPCP
YLRSADTTHSTVLGLYNTLNPEASASPCCVPQDLEPLTILYYVGRTPKVE
QLSNMVVKSCKCS.

Suitably, the secreted factor may comprise a variant of SEQ ID NO: 24.

Suitably, the secreted factor may comprise a variant of SEQ ID NO: 19, SEQ ID NO: 20, SEQ ID NO: 23 or SEQ ID NO: 24, wherein the variant has less than 100% sequence identity to SEQ ID NO: 19, SEQ ID NO: 20, SEQ ID NO: 23 or SEQ ID NO: 24. Suitably, the variant may have at least 80%, at least 85%, at least 90%, at least 95%, at least 98% or 99% sequence identity to SEQ ID NO: 19, SEQ ID NO: 20, SEQ ID NO: 23 or SEQ ID NO: 24.

### Mutant TGFβ

In one embodiment, the secreted factor comprises a mutant TGFβ.

The mutant TGFβ is capable of binding a transforming growth factor beta receptor (TβR) and disrupting its interaction with transforming growth factor beta (TGFβ).

As used herein a “mutant TGFβ” is a TGFβ protein which comprises one or more amino acid mutations with respect to the wild-type TGFβ polypeptide.

Suitably, a mutant TGFβ may bind TβR1 or TβRII and prevent the approximation of both receptors, thereby inhibiting signalling downstream of TβR.

Suitably, a mutant TGFβ may comprise a mutation which eliminates or decreases their ability to signal through the ALKS (TβRI) receptor. Such mutants maintain their ability to bind with high affinity to TβRII and TβRIII receptors, but are unable to signal because they do not interact with the ALKS (TβRI) receptor, negatively modulating wild-type TGFβ signalling by competing with them for binding to high affinity TβRII and TβRIII receptors.

Suitably, the mutated TGFβ maintains a binding affinity to TβRII of at least the binding affinity of wild-type TGFβ1 and TGFβ3.

Suitably, the mutant TGFβ comprises mutations which increase their affinity for the receptor TβRII and/or TβRIII. For example, the mutation may be at the interaction interface with the receptor.

Suitably, the mutant TGFβ may comprise one or more mutations with respect to SEQ ID NO: 19. Suitably, the mutant TGFβ may comprise one or more mutations with respect to SEQ ID NO: 20. Suitably, the mutant TGFβ may comprise one or more mutations with respect to SEQ ID NO: 23. Suitably, the mutant TGFβ may comprise one or more mutations with respect to SEQ ID NO: 24.

For example, the mutant TGFβ may comprise one, two, three, four five or six mutations.

Suitably the mutations are selected to have different physicochemical properties with respect to the amino acid present in the wild-type polypeptide. For example, the mutation may be a change in an amino acid residue from nonpolar to polar, from charged to uncharged from large to small or from acid to basic.

Suitably, the mutant TGFβ may comprise one or more mutations at positions selected from amino acid residues: 30, 43, 101, 51, 67 or 6; when the amino acid number is determined by alignment with SEQ ID NO: 23. In other words, the amino acid numbering corresponds with the numbering of amino acids as set forth in SEQ ID NO: 23.

Suitably, the mutant TGFβ may comprise one or more mutations at positions selected from amino acid residues: W30, W32, L101, L51, Q67 and Y6 when the amino acid number is determined by alignment with SEQ ID NO: 23. These amino acid residues of TGFβ have been identified as important for TβRI interaction and important in stable binding.

Suitably, the mutant TGFβ may comprise one or more mutations, for example two, three, four, five or six mutations, at amino acid residues W30, W32, L101, L51, Q67 and Y6 when the amino acid number is determined by alignment with SEQ ID NO: 23, wherein:

amino acid residue 30 is mutated to N, R, K, D, Q, L, S, P, V, I, G, C, T, A or E; and/or

amino acid residue 32 is mutated to A; and/or

amino acid residue 101 is mutated to A, E; and/or

amino acid residue 51 is mutated to Q, W, Y, A; and/or

amino acid residue 67 is mutated to H, F, Y, W, Y; and/or

amino acid residue 6 is mutated to A.

Suitably, the mutant TGFβ may comprise mutations at each of amino acid residues W30, W32, L101, L51, Q67 and Y6 when the amino acid number is determined by alignment with SEQ ID NO: 23. Suitably, the amino acid substitutions may be selected from the list above.

Suitably, the mutant TGFβ may comprise the following mutations: W30E, L101E and L51Q when the amino acid number is determined by alignment with SEQ ID NO: 23.

Suitably, the mutant TGFβ may comprise the following mutations: W30E, L101A and L51Q when the amino acid number is determined by alignment with SEQ ID NO: 23.

Suitably, the mutant TGFβ may comprise the following mutations: W30E, L101E, L51Q and Q67H when the amino acid number is determined by alignment with SEQ ID NO: 23.

Suitably, the mutant TGFβ may comprise the following mutations: W30E, L101A, K97D, E12H, L51Q and Q67H when the amino acid number is determined by alignment with SEQ ID NO: 23.

In one aspect, the secreted factor comprises a truncated TGFβ polypeptide.

Suitably, the truncation may comprise an N-terminal deletion with respect to a wild-type TGFβ polypeptide. Suitably, the truncation may comprise a C-terminal deletion with respect to a wild-type polypeptide. The truncation may comprise a deletion of 1, 2, 3, 4, 5, 10, 15, 20, 25, 30, 35, 40, 45 or 50 amino acids.

In one aspect, the secreted factor is monomeric TGFβ.

Suitably, the secreted factor may be unable to homodimerize, thereby inhibiting signalling downstream of TβR. Suitably, the secreted factor may prevent association between TβRI and TβRII receptors.

Suitably, the secreted factor may be a TGFβ which lacks the heel helix α3, a structural motif essential for binding the TGFβ type I receptor (TβRI) but dispensable for binding TβRII. Suitably, the secreted factor may be a TGFβ which lacks the heel helix α3, a structural motif essential for binding the TGFβ type I receptor (TβRI) but dispensable for binding TβRII and which lacks Cys-77.

The amino acid sequence of a TGFβ monomer is set forth in SEQ ID NO: 25. SEQ ID NO: 25 comprises a signal peptide and a latency associated peptide (LAP).

(SEQ ID NO: 25)
MHYCVLSAFLILHLVTVALSLSTCSTLDMDQFMRKRIEAIRGQILSKLKL
TSPPEDYPEPEEVPPEVISIYNSTRDLLQEKASRRAAACERERSDEEYYA
KEVYKIDMPPFFPSENAIPPTFYRPYFRIVRFDVSAMEKNASNLVKAEFR
VFRLQNPKARVPEQRIELYQILKSKDLTSPTQRYIDSKVVKTRAEGEWLS
FDVTDAVHEWLHHKDRNLGFKISLHCPCCTFVPSNNYIIPNKSEELEARF
AGIDGTSTYTSGDQKTIKSTRKKNSGKTPHLLLMLLPSYRLESQQTNRRK
KRALDAAYCFRNVQDNCCLRPLYIDFRKDLGWKWIHEPKGYNANFCAGAC
PYRASKSPSCVSQDLEPLTIVYYVGRKPKVEQLSNMIVKSCKCS.

Suitably, the secreted factor may comprise an amino acid sequence set forth in SEQ ID NO: 25 or a variant thereof. The variant TGFβ monomer may have at least 80%, 85%, 90%, 95%, 98% or 99% sequence identity to SEQ ID NO: 25, provided that the polypeptide provides a monomer which is capable of binding a transforming growth factor beta receptor (TβR) and disrupting its interaction with transforming growth factor beta (TGFβ).

Suitably, the secreted factor may comprise or consist of the amino acid sequence set forth in SEQ ID NO: 25.

The amino acid sequence of the signal peptide comprised within SEQ ID NO: 25 is set forth in SEQ ID NO: 26.

(SEQ ID NO: 26)
MHYCVLSAFLILHLVTVALSALDAAYCFRNVQDNCCLRPLYIDFRKDLGW
KWIHEPKGYNANFCAGACPYRASKSPSCVSQDLEPLTIVYYVGRKPKVEQ
LSNMIVKSCKCS.

Suitably, the signal peptide may comprise the amino acid sequence set forth in SEQ ID NO: 26 or a variant thereof. The variant signal peptide may have at least 80%, 85%, 90%, 95%, 98% or 99% sequence identity to SEQ ID NO: 26, provided that the sequence provides an effective signal peptide. That is, provided that the sequence is capable of directing a newly synthesized secreted factor to the secretory pathway.

In another embodiment, the secreted factor may be an antibody or a variant thereof.

Suitably, the antibody may be a full-length antibody, a single chain antibody fragment, a F(ab) fragment, a F(ab′)₂ fragment, a F(ab′) fragment, a single domain antibody (sdAb), a VHH/nanobody, a nanobody, an affibody, a fibronectin artificial antibody scaffold, an anticalin, an affilin, a DARPin, a VNAR, an iBody, an affimer, a fynomer, a domain antibody (DAb), an abdurin/nanoantibody, a centyrin, an alphabody or a nanofitin which is capable of binding a TβR and disrupting its interaction with TGFβ.

TGFβ antibodies are well known in the art.

A variant secreted factor according to the present invention may have, for example, one, two or three or more amino acid mutations, for example one, two or three or more amino acid substitutions with respect to the amino acid sequences of secreted factors disclosed herein, for example in SEQ ID NO: 25 or SEQ ID NO: 26. Preferably, the amino acid substitutions are conservative substitutions.

Conservative amino acid substitutions may be made on the basis of similarity in polarity, charge, solubility, hydrophobicity, hydrophilicity, and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to Table 2 below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The present invention also encompasses homologous substitution (substitution and replacement are both used herein to mean the interchange of an existing amino acid residue, with an alternative residue) i.e. like-for-like substitution such as basic for basic, acidic for acidic, polar for polar etc.

Unless otherwise explicitly stated herein by way of reference to a specific, individual amino acid, amino acids may be substituted using conservative substitutions as recited below.

An aliphatic, non-polar amino acid may be a glycine, alanine, proline, isoleucine, leucine or valine residue. An aliphatic, polar uncharged amino may be a cysteine, serine, threonine, methionine, asparagine or glutamine residue. An aliphatic, polar charged amino acid may be an aspartic acid, glutamic acid, lysine or arginine residue. An aromatic amino acid may be a histidine, phenylalanine, tryptophan or tyrosine residue.

Suitably, a conservative substitution may be made between amino acids in the same line in Table 2.

Variants (e.g. mutants) may maintain their ability to bind to TβRII. The ability of a variant TGFβ to bind to a TβRII may be measured by any means known in the art for example by an ELISA assay, to detect TβRII receptor chain.

Variant TGFβ. (e.g. mutant TGFβ) may block the binding of wild-type TGFβ ligands to TβRII. This may be measured by a competitive ELISA covering the plates with TGFβ ligand and assessing the ability of the variant to inhibit TβRII binding to the ligands.

Variant TGFβ. (e.g. mutant TGFβ) may have reduced ability to signal through TβRI or may not be capable of signalling through TβRI. This may be measured by western blotting and quantifying the levels and ratios of phosphorylated SMAD2 and SMAD3. Mutant TGFβ may induce 100 times less phosphorylation than wild-type TGFβ.

Variant TGFβ. (e.g. mutated TGFβ) may be are capable of inhibiting signalling induced by wild-type TGFβ. This may be measured by Western immunoblotting assays and quantifying the levels of phosphorylated SMAD2 and SMAD3 in cell lysates treated with the mutant or wild-type TGFβ.

### Antibody

The present invention provides cell which comprises a chimeric antigen receptor (CAR) or transgenic T cell receptor (TCR) and secretes an antibody which binds a transforming growth factor beta receptor (TβR). The antibody may be capable of binding a transforming growth factor beta receptor (TβR) and disrupting its interaction with transforming growth factor beta (TGFβ).

The antibody may be a full-length antibody or a fragment thereof such as a single chain antibody fragment, a F(ab) fragment, a F(ab′)2 fragment, a F(ab′) fragment, a domain antibody (dAb), a VHH/nanobody, a nanobody, a VNAR or IgNAR.

The term “antibody” includes antibody mimetics such as an affibody, a fibronectin artificial antibody scaffold, an anticalin, an affilin, a DARPin, an iBody, an affimer, a fynomer, an abdurin/nanoantibody, a centyrin, an alphabody a nanofitin or a D-domain which are capable of binding a transforming growth factor beta receptor (TβR) and disrupting its interaction with transforming growth factor beta (TGFβ).

The antibody may be a dAb comprising one of the following sets of complementarity determining regions (CDRs):

(SEQ ID No. 1)
a) CDR1-GIDLSTKS;
(SEQ ID No. 2)
CDR2-HSSSGGTT;
and
(SEQ ID No. 3)
CDR3-TAPVGY;
(SEQ ID No. 4)
b) CDR1-GIILSSKA;
(SEQ ID No. 5)
CDR2-HSSVSGTT;
and
(SEQ ID No. 6)
CDR3-TAPVGH;
(SEQ ID No. 7)
c) CDR1-GITFSTKP;
(SEQ ID No. 8)
CDR2-HISSDGTT;
and
(SEQ ID No. 9)
CDR3-TAPVGY;
or
(SEQ ID No. 10)
d) CDR1-GRSFSIYG;
(SEQ ID No. 11)
CDR2-ITGSGLTT;
and
(SEQ ID No. 12)
CDR3-AATRAYSGYYGENPTRYAY.

The dAb may comprise one of the sequences shown as SEQ ID No. 13, 14, 15 or 16.

SEQ ID No. 13
QVQLQESGGGLVQAGGSLRLSCAASGIDLSTKSVAVVYRQPPGKQREGVA
HSSSGGTTIYADSVKGRFTISRDNSKNTVYLEMNSLKPEDTAVYYCTAPV
GYWGQGTQVTVS
SEQ ID No. 14
QVQLQESGGGLVQPGGSLRLSCAASGIILSSKAVAVVYRQPPGQQREGVA
HSSVSGTTIYADSVKGRFTVSRDNAKNTVYLEMNSLKPEDTAVYYCTAPV
GHWGQGTQVTVS
SEQ ID No. 15
QVQLQQSGGGLVQAGGSLRLSCAASGITFSTKPVAVVYRQPPGKQREGVA
HISSDGTTIYADSVKGRFTVSRDNAKNTVYLEMNSLKPEDTGVYYCTAPV
GYWGQGTQVTVS
SEQ ID No. 16
QVQLQQSGGGLVQAGGSLRLACAASGRSFSIYGITWFRQAPGKEREFVAA
ITGSGLTTNYADSVKGRFTISRDNAKNMVYLQLNSLKPDDTAVYYCAATR
AYSGYYGENPTRYAYWGQGTQVTVS

The antibody may comprise a variant of SEQ ID No. 13 to 16. Variant sequences may have at least 80%, 85%, 90%, 95%, 98% or 99% sequence identity to SEQ ID NO: 13, 14, 15 or 16. The variant sequence may comprise one of the sets of CDRs labelled a), b), c) or d) above. The variant antibody should retain the capacity to bind a transforming growth factor beta receptor (TβR)

### Tuning

Secretion of the TβR-binding secreted factor or antibody by the cell of the invention may be “tunable”.

As used herein, “tunable” means that it is possible to increase, decrease, turn on or turn off secretion or activity of the secreted factor or antibody.

Production of the a secreted factor or antibody may be controlled or tuned by an inducible promoter. For example, production of the a secreted factor or antibody may be regulated by Nuclear factor of activated T cells (NFAT) response element.

An NFAT response element may comprise the nucleotide sequence set forth in SEQ ID NO: 27 or a variant thereof.

(SEQ ID NO: 27)
GGAGGAAAAACTGTTTCATACAGAAGGCGT.

Variant sequences of SEQ ID NO: 27 may have at least 80%, 85%, 90%, 95%, 98% or 99% sequence identity to SEQ ID NO: 27. Suitably, the variant sequence is able to function as a NFAT response element.

The NFAT response element may comprise repeat units such as 3, 4, 5 or 6 repeat units. Suitably, the NFAT response element may comprise 3, 4, 5 or 6 repeat units of SEQ ID NO: 27. The NFAT response element may be positioned in front of a promoter (e.g. a CMV promoter).

Secretion of the secreted factor or antibody may be controlled or tuned through interaction with an intracellular retention domain. An agent may be used to disrupt the interaction with the intracellular retention domain, thereby allowing secretion of the secreted factor or antibody from the cell.

Alternatively or in addition, the activity of the secreted factor or antibody may be controlled or tuned.

“Activity” as used herein means the ability of the secreted factor or antibody to bind a transforming growth factor beta receptor (TβR) thereby disrupting its interaction with transforming growth factor beta (TGFβ).

For example, the activity of the secreted factor or antibody may be inhibited by providing a sink which binds the factor, preventing or reducing interaction between the secreted factor or antibody and the receptor. For example, blockade of the secreted factor or antibody with antibodies or soluble TβR may be used to control or tune the activity of the antibody.

### Binding

As used herein, “disrupted” or “disruption” means that the binding between TβR and TGFβ is reduced or eliminated completely by the secreted factor or antibody.

For example, binding between TβR and TGFβ may be reduced by at least 10%, at least 20%, at least 30%, at least 40%, at least 50%, at least 60%, at least 70%, at least 80%, at least 90%, at least 95% or at least 99% by the secreted factor or antibody. Suitably, binding between TβR and TGFβ may be eliminated.

The interaction between TβR and TGFβ may be disrupted by the secreted factor or antibody binding competitively to TβR.

As used herein competitive binding of the secreted factor or antibody refers to the binding of a secreted factor or antibody which prevents binding between TβR and TGFβ. The secreted factor or antibody may bind competitively by directly binding to binding site of TβR which interacts with the reciprocal binding site on TGFβ. Alternatively; the secreted factor or antibody may bind competitively by binding to a region which overlaps with the binding site of TβR which interacts with the reciprocal binding site on TGFβ.

The secreted factor or antibody may be capable of specifically binding TβR at a higher affinity than the binding between the TβR and TGFβ.

As used herein, “higher affinity” means that the secreted factor or antibody binds to TβR with at least 5, 10, 20, 50, 100, 1000 or 10000-fold greater affinity than the binding affinity between TβR and TGFβ.

Assays for measuring binding affinity and competitive binding are known in the art such as radioactive ligand binding assays (including saturation binding, scatchard plot), non-radioactive ligand binding assays (including fluorescence polarization, fluorescence resonance energy transfer and surface plasmon resonance/Biacore, and solid phase ligand binding assays. Any method known in the art may be used to measure binding affinity of the antibody.

Binding of the secreted factor or antibody may prevent the approximation of WI and TβRII.

Binding of the secreted factor or antibody may reduce or eliminate signalling downstream of TβR. Suitably, signalling downstream of TβR may be reduced by at least 10%, at least 20%, at least 30%, at least 40%, at least 50%, at least 60%, at least 70%, at least 80%, at least 90%, at least 95% or at least 99% by the secreted factor. Suitably, signalling downstream of TβR may be eliminated.

Assays for measuring downstream signalling of TβR are known in the art such as luminescent kinase assays which measure ADP formed from the kinase reaction or measuring the proportion of cytoplasmic signalling molecules such as SMAD/SMAD2 phosphorylation. Any method known in the art may be used to measure downstream signalling of TβR.

### Cell

The present invention relates to cell which comprises a chimeric antigen receptor (CAR) or transgenic T cell receptor (TCR) and secretes an secreted factor or antibody which binds a transforming growth factor beta receptor (TβR).

The cell is “engineered” in that it has been modified to comprise or express a nucleic acid sequence which is not naturally encoded by the cell. Methods for engineering cells are known in the art and include but are not limited to genetic modification of cells e.g. by transduction such as retroviral or lentiviral transduction, transfection (such as transient transfection—DNA or RNA based) including lipofection, polyethylene glycol, calcium phosphate and electroporation. Any suitable method may be used to introduce a nucleic acid sequence into a cell.

Accordingly, the nucleic acid sequence encoding secreted factor which is capable of binding a transforming growth factor beta receptor (TβR) and disrupting its interaction with transforming growth factor beta (TGFβ) is not naturally expressed by a corresponding, unmodified cell.

Engineered cells according to the present invention may be generated by introducing DNA or RNA coding a CAR, TCR and/or antibody by one of many means including transduction with a viral vector, transfection with DNA or RNA.

Cells may be activated and/or expanded prior to the transfection or transduction, for example by treatment with an anti-CD3 monoclonal antibody or both anti-CD3 and anti-CD28 monoclonal antibodies. As used herein “activated” means that a cell has been stimulated, causing the cell to proliferate, differentiate or initiate an effector function.

Methods for measuring cell activation are known in the art and include, for example, measuring the expression of activation markers by flow cytometry, such as the expression of CD69, CD25, CD38 or HLA-DR or measuring intracellular cytokines.

As used herein “expanded” means that a cell or population of cells has been induced to proliferate.

The expansion of a population of cells may be measured for example by counting the number of cells present in a population. The phenotype of the cells may be determined by methods known in the art such as flow cytometry.

The cell may be an “immune effector cell” which is a cell of the immune system which responds to a stimulus and effects a change.

Immune effector cells include T cells (such as an alpha-beta T cell or a gamma-delta T cell), a B cells (such as a plasma cell), a Natural Killer (NK) cells or a macrophages.

“Cytolytic immune cell” as used herein is a cell which directly kills other cells. Cytolytic cells may kill cancerous cells; virally infected cells or other damaged cells. Cytolytic immune cells include T cells and Natural killer (NK) cells.

Cytolytic immune cells can be T cells or T lymphocytes which are a type of lymphocyte that play a central role in cell-mediated immunity. T cells can be distinguished from other lymphocytes, such as B cells and NK cells, by the presence of a TCR on their cell surface.

Cytolytic T cells (TC cells, or CTLs) destroy virally infected cells and tumour cells, and are also implicated in transplant rejection. CTLs express the CD8 at their surface. CTLs may be known as CD8+ T cells. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Suitably, the cell of the present invention may be a T-cell. Suitably, the T cell may be an alpha-beta T cell. Suitably, the T cell may be a gamma-delta T cell.

Natural Killer Cells (or NK cells) are a type of cytolytic cell which form part of the innate immune system. NK cells provide rapid responses to innate signals from virally infected cells in an MHC independent manner.

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

Suitably, the cell of the present invention may be a wild-type killer (NK) cell. Suitably, the cell of the present invention may be a cytokine induced killer cell.

The cell may be derived from a patient's own peripheral blood (1st party), or in the setting of a haematopoietic stem cell transplant from donor peripheral blood (2nd party), or peripheral blood from an unconnected donor (3rd party). T or NK cells, for example, may be activated and/or expanded prior to being transduced with nucleic acid molecule(s) encoding the polypeptides of the invention, for example by treatment with an anti-CD3 monoclonal antibody.

Alternatively, the cell may be derived from ex vivo differentiation of inducible progenitor cells or embryonic progenitor cells to T cells. Alternatively, an immortalized T-cell line which retains its lytic function may be used.

The cell may be a haematopoietic stem cell (HSC). HSCs can be obtained for transplant from the bone marrow of a suitably matched donor, by leukapheresis of peripheral blood after mobilization by administration of pharmacological doses of cytokines such as G-CSF [peripheral blood stem cells (PBSCs)], or from the umbilical cord blood (UCB) collected from the placenta after delivery. The marrow, PBSCs, or UCB may be transplanted without processing, or the HSCs may be enriched by immune selection with a monoclonal antibody to the CD34 surface antigen.

### Chimeric Antigen Receptor (CAR)

The cell of the present invention may express a chimeric antigen receptor (CAR).

Classical CARs are chimeric type I trans-membrane proteins which connect an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site or on a ligand for the target antigen. A spacer domain may be necessary to isolate the binder from the membrane and to allow it a suitable orientation. A common spacer domain used is the Fc of IgG1. More compact spacers can suffice e.g. the stalk from CD8a and even just the IgG1 hinge alone, depending on the antigen. A trans-membrane domain anchors the protein in the cell membrane and connects the spacer to the endodomain.

Early CAR designs had endodomains derived from the intracellular parts of either the γ chain of the FcεR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 4-1BB which transmit survival signals. Even more potent third generation CARs have now been described which have endodomains capable of transmitting activation, proliferation and survival signals.

CAR-encoding nucleic acids may be transferred to T cells using, for example, retroviral vectors. In this way, a large number of antigen-specific T cells can be generated for adoptive cell transfer. When the CAR binds the target-antigen, this results in the transmission of an activating signal to the T-cell it is expressed on. Thus the CAR directs the specificity and cytotoxicity of the T cell towards cells expressing the targeted antigen.

### Antigen Binding Domain

The antigen-binding domain is the portion of a CAR which recognizes antigen.

Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a wild-type ligand of the target antigen; a peptide with sufficient affinity for the target; a single domain binder such as a camelid; an artificial binder single as a Darpin; or a single-chain derived from a T-cell receptor.

CARs have also been described in which the antigen-binding domain is based on a ligand for the target antigen. For example, WO2015/052538 describes a BCMA-specific CAR in which the binding domain is based on a proliferation-inducing ligand (APRIL), rather than a BCMA-binding antibody.

The antigen-binding domain may bind to a tumour associated antigens (TAA). Various TAAs have been described as being potentially suitable for targeting with a CAR or engineered TCR for cancer therapy, some of which are shown in the following Table 1.

TABLE 1

| Cancer type | TAA |
|---|---|
| Diffuse Large B-cell Lymphoma | CD19, CD20, CD22 |
| Breast cancer | ErbB2, MUC1 |
| AML | CD13, CD33 |
| Neuroblastoma | GD2, NCAM, ALK, GD2 |
| B-CLL | CD19, CD52, CD160 |
| Colorectal cancer | Folate binding protein, CA-125 |
| Chronic Lymphocytic Leukaemia | CD5, CD19 |
| Glioma | EGFR, Vimentin |
| Multiple myeloma | BCMA, CD138, TACI |
| Renal Cell Carcinoma | Carbonic anhydrase IX, G250 |
| Prostate cancer | PSMA, PSCA |
| Bowel cancer | A33 |

### Transmembrane Domain

The transmembrane domain is the sequence of a CAR that spans the membrane. It may comprise a hydrophobic alpha helix. The transmembrane domain may be derived from CD28, which gives good receptor stability.

### CAR or TCR Signal Peptide

The CAR or transgenic TCR expressed by the cell of the present invention may comprise a signal peptide so that when it is expressed in a cell, such as a T-cell, the nascent protein is directed to the endoplasmic reticulum and subsequently to the cell surface, where it is expressed.

The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

### Spacer Domain

The CAR may comprise a spacer sequence to connect the antigen-binding domain with the transmembrane domain. A flexible spacer allows the antigen-binding domain to orient in different directions to facilitate binding.

The spacer sequence may, for example, comprise an IgG1 Fc region, an IgG1 hinge or a human CD8 stalk or the mouse CD8 stalk. The spacer may alternatively comprise an alternative linker sequence which has similar length and/or domain spacing properties as an IgG1 Fc region, an IgG1 hinge or a CD8 stalk. A human IgG1 spacer may be altered to remove Fc binding motifs.

### Intracellular Signalling Domain

The intracellular signalling domain is the signal-transmission portion of the CAR.

The most commonly used signalling domain component is that of CD3-zeta endodomain, which contains 3 ITAMs. This transmits an activation signal to the T cell after antigen is bound. CD3-zeta may not provide a fully competent activation signal and additional co-stimulatory signalling may be needed. For example, chimeric CD28 and OX40 can be used with CD3-Zeta to transmit a proliferative/survival signal, or all three can be used together.

The intracellular signalling domain may be or comprise a T cell signalling domain.

The intracellular signalling domain may comprise one or more immunoreceptor tyrosine-based activation motifs (ITAMs). An ITAM is a conserved sequence of four amino acids that is repeated twice in the cytoplasmic tails of certain cell surface proteins of the immune system. The motif contains a tyrosine separated from a leucine or isoleucine by any two other amino acids, giving the signature YxxL/I. Two of these signatures are typically separated by between 6 and 8 amino acids in the tail of the molecule (YxxL/Ix₍₆₋₈₎YxxL/I).

ITAMs are important for signal transduction in immune cells. Hence, they are found in the tails of important cell signalling molecules such as the CD3 and ζ-chains of the T cell receptor complex, the CD79 alpha and beta chains of the B cell receptor complex, and certain Fc receptors. The tyrosine residues within these motifs become phosphorylated following interaction of the receptor molecules with their ligands and form docking sites for other proteins involved in the signalling pathways of the cell.

The intracellular signalling domain component may comprise, consist essentially of, or consist of the CD3-ζ endodomain, which contains three ITAMs. Classically, the CD3-ζ endodomain transmits an activation signal to the T cell after antigen is bound.

The intracellular signalling domain may comprise additional co-stimulatory signalling. For example, 4-1BB (also known as CD137) can be used with CD3-ζ, or CD28 and OX40 can be used with CD3-ζ to transmit a proliferative/survival signal.

The endodomain may comprise:

(i) an ITAM-containing endodomain, such as the endodomain from CD3 zeta; and/or

(ii) a co-stimulatory domain, such as the endodomain from CD28 or ICOS; and/or

(iii) a domain which transmits a survival signal, for example a TNF receptor family endodomain such as OX-40, 4-1BB, CD27 or GITR.

A number of systems have been described in which the antigen recognition portion is on a separate molecule from the signal transmission portion, such as those described in WO015/150771; WO2016/124930 and WO2016/030691. The CAR of the cell of the present invention may therefore comprise an antigen-binding component comprising an antigen-binding domain and a transmembrane domain; which is capable of interacting with a separate intracellular signalling component comprising a signalling domain. The vector of the invention may express a chimeric receptor signalling system comprising such an antigen-binding component and intracellular signalling component.

### Transgenic T-Cell Receptor (TCR)

The cell of the invention may express a transgenic T-cell receptor (TCR).

The TCR is a molecule found on the surface of T cells which is responsible for recognizing fragments of antigen as peptides bound to major histocompatibility complex (MHC) molecules.

The TCR is a heterodimer composed of two different protein chains. In humans, in 95% of T cells the TCR consists of an alpha (α) chain and a beta (β) chain (encoded by TRA and TRB, respectively), whereas in 5% of T cells the TCR consists of gamma and delta (γ/δ) chains (encoded by TRG and TRD, respectively).

When the TCR engages with antigenic peptide and MHC (peptide/MHC), the T lymphocyte is activated through signal transduction.

In contrast to conventional antibody-directed target antigens, antigens recognized by the TCR can include the entire array of potential intracellular proteins, which are processed and delivered to the cell surface as a peptide/MHC complex.

It is possible to engineer cells to express heterologous (i.e. non-native) TCR molecules by artificially introducing the TRA and TRB genes; or TRG and TRD genes into the cell using a vector. For example the genes for engineered TCRs may be reintroduced into autologous T cells and transferred back into patients for T cell adoptive therapies. Such ‘heterologous’ TCRs may also be referred to herein as ‘transgenic TCRs’.

The transgenic TCR for use in the present invention may recognise a tumour associated antigen (TAA) when fragments of the antigen are complexed with major histocompatibility complex (MHC) molecules on the surface of another cell.

Suitably, the transgenic TCR for use in the present invention may recognise a TAA listed in Table 1.

### Nucleic Acid Sequence

The present invention provides a nucleic acid sequence which encodes a domain antibody (dAb) which binds transforming growth factor beta receptor type II (TβRII) and comprises one of the following sets of complementarity determining regions (CDRs):

(SEQ ID No. 1)
a) CDR1-GIDLSTKS;
(SEQ ID No. 2)
CDR2-HSSSGGTT;
and
(SEQ ID No. 3)
CDR3-TAPVGY;
(SEQ ID No. 4)
b) CDR1-GIILSSKA;
(SEQ ID No. 5)
CDR2-HSSVSGTT;
and
(SEQ ID No. 6)
CDR3-TAPVGH;
(SEQ ID No. 7)
c) CDR1-GITFSTKP;
(SEQ ID No. 8)
CDR2-HISSDGTT;
and
(SEQ ID No. 9)
CDR3-TAPVGY;
or
(SEQ ID No. 10)
d) CDR1-GRSFSIYG;
(SEQ ID No. 11)
CDR2-ITGSGLTT;
and
(SEQ ID No. 12)
CDR3-AATRAYSGYYGENPTRYAY.

The nucleic acid sequence may encode a dAb which comprises one of the sequences shown as SEQ ID No. 13, 14, 15 or 16.

### Nucleic Acid Construct/Kit of Nucleic Acid Sequences

The present invention provides a nucleic acid construct which comprises:

(i) a first nucleic acid sequence encoding a secreted factor or antibody which is capable of binding a transforming growth factor beta receptor (TβR); and

(ii) a second nucleic acid sequence which encodes a CAR or transgenic TCR.

The present invention also provides a kit comprising nucleic acid sequences according to the present invention. For example, the kit may comprise

(i) a first nucleic acid sequence encoding a secreted factor or antibody which is capable of binding a transforming growth factor beta receptor (TβR); and

(ii) a second nucleic acid sequence which encodes a CAR or transgenic TCR.

As used herein, the terms “polynucleotide”, “nucleotide”, and “nucleic acid” are intended to be synonymous with each other.

Suitably, the nucleic acid construct may comprise a plurality of nucleic acid sequences which encode a secreted factor or antibody which is capable of binding a transforming growth factor beta receptor (TβR); and a CAR or transgenic TCR. For example, the nucleic acid construct may comprise two, three, four or more nucleic acid sequences which encode different components of the invention. Suitably, the plurality of nucleic acid sequences may be separated by co-expression sites as defined herein.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described herein to reflect the codon usage of any particular host organism in which the polypeptides are to be expressed. Suitably, the polynucleotides of the present invention are codon optimised to enable expression in a mammalian cell, in particular an immune effector cell as described herein.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence or amino acid sequence includes any substitution of, variation of, modification of, replacement of, deletion of or addition of one (or more) nucleic acid(s) from or to the sequence.

### Co-Expression Site

A co-expression site is used herein to refer to a nucleic acid sequence enabling co-expression of nucleic acid sequences encoding a secreted factor or antibody which is capable of binding a transforming growth factor beta receptor (TβR) and a CAR or transgenic TCR according to the present invention.

Suitably, there may be a co-expression site between the first nucleic acid sequence and the second nucleic acid sequence. Suitably, there may be a co-expression site between the nucleic acid sequence encoding the secreted factor or antibody and the nucleic acid sequence which encodes the CAR or transgenic TCR.

Suitably, in embodiments where a plurality of co-expression sites is present in the engineered polynucleotide, the same co-expression site may be used.

The co-expression site may be a cleavage site. The cleavage site may be any sequence which enables the two polypeptides to become separated. The cleavage site may be self-cleaving, such that when the polypeptide is produced, it is immediately cleaved into individual peptides without the need for any external cleavage activity.

The term “cleavage” is used herein for convenience, but the cleavage site may cause the peptides to separate into individual entities by a mechanism other than classical cleavage. For example, for the Foot-and-Mouth disease virus (FMDV) 2A self-cleaving peptide (see below), various models have been proposed for to account for the “cleavage” activity: proteolysis by a host-cell proteinase, autoproteolysis or a translational effect (Donnelly et al (2001) J. Gen. Virol. 82:1027-1041). The exact mechanism of such “cleavage” is not important for the purposes of the present invention, as long as the cleavage site, when positioned between nucleic acid sequences which encode proteins, causes the proteins to be expressed as separate entities.

The cleavage site may be a furin cleavage site. Furin is an enzyme which belongs to the subtilisin-like proprotein convertase family. The members of this family are proprotein convertases that process latent precursor proteins into their biologically active products. Furin is a calcium-dependent serine endoprotease that can efficiently cleave precursor proteins at their paired basic amino acid processing sites. Examples of furin substrates include proparathyroid hormone, transforming growth factor beta 1 precursor, proalbumin, pro-beta-secretase, membrane type-1 matrix metalloproteinase, beta subunit of pro-nerve growth factor and von Willebrand factor. Furin cleaves proteins just downstream of a basic amino acid target sequence (canonically, Arg-X-(Arg/Lys)-Arg′) and is enriched in the Golgi apparatus.

The cleavage site may be a Tobacco Etch Virus (TEV) cleavage site.

TEV protease is a highly sequence-specific cysteine protease which is chymotrypsin-like proteases. It is very specific for its target cleavage site and is therefore frequently used for the controlled cleavage of fusion proteins both in vitro and in vivo. The consensus TEV cleavage site is ENLYFQ\S (where ‘\’ denotes the cleaved peptide bond). Mammalian cells, such as human cells, do not express TEV protease. Thus in embodiments in which the present nucleic acid construct comprises a TEV cleavage site and is expressed in a mammalian cell—exogenous TEV protease must also expressed in the mammalian cell.

The cleavage site may encode a self-cleaving peptide. A ‘self-cleaving peptide’ refers to a peptide which functions such that when the polypeptide comprising the proteins and the self-cleaving peptide is produced, it is immediately “cleaved” or separated into distinct and discrete first and second polypeptides without the need for any external cleavage activity.

The self-cleaving peptide may be a 2A self-cleaving peptide from an aphtho- or a cardiovirus. The primary 2A/2B cleavage of the aptho- and cardioviruses is mediated by 2A “cleaving” at its own C-terminus. In apthoviruses, such as foot-and-mouth disease viruses (FMDV) and equine rhinitis A virus, the 2A region is a short section of about 18 amino acids, which, together with the N-terminal residue of protein 2B (a conserved proline residue) represents an autonomous element capable of mediating “cleavage” at its own C-terminus (Donelly et al (2001) as above).

“2A-like” sequences have been found in picornaviruses other than aptho- or cardioviruses, ‘picornavirus-like’ insect viruses, type C rotaviruses and repeated sequences within Trypanosoma spp and a bacterial sequence (Donnelly et al., 2001) as above.

The co-expression sequence may be an internal ribosome entry sequence (IRES). The co-expressing sequence may be an internal promoter.

### Vector

The present invention also provides a vector, or kit of vectors which comprises one or more nucleic acid sequence(s) or nucleic acid construct(s) of the invention. Such a vector may be used to introduce the nucleic acid sequence(s) or construct(s) into a host cell so that it expresses a secreted factor or antibody and a CAR or transgenic TCR as defined herein.

Suitably, the vector may comprise a plurality of nucleic acid sequences which encode different components as provided by the present invention. For example, the vector may comprise two, three, four or more nucleic acid sequences which encode different components, such as the secreted factor or antibody which binds TβR and a CAR or transgenic TCR. Suitably, the plurality of nucleic acid sequences may be separated by co-expression sites as defined herein.

The vector may, for example, be a plasmid or a viral vector, such as a retroviral vector or a lentiviral vector, or a transposon based vector or synthetic mRNA.

The vector may be capable of transfecting or transducing a cell.

### Pharmaceutical Composition

The present invention also relates to a pharmaceutical composition comprising a cell according to the present invention or a cell obtainable (e.g. obtained) by a method according to the present invention.

The present invention also provides a pharmaceutical composition comprising a domain antibody (dAb), cell or plurality of cells as defined herein.

In particular, the invention relates to a pharmaceutical composition containing a cell according to the present invention.

The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

### Method of Treatment

The present invention provides a method for treating and/or preventing a disease which comprises the step of administering a cell or plurality of cells or pharmaceutical composition according to the invention to a subject.

Suitably, the present methods for treating and/or preventing a disease may comprise administering a cell according to the present invention (for example in a pharmaceutical composition as described above) to a subject.

The present invention also provides a method for treating and/or preventing a disease in a subject which subject comprises cells of the invention, which method comprises the step of administering an agent to the subject wherein the agent is capable of controlling the secretion or activity of secreted factor or antibody. As such, this method involves administering an agent to a subject which already comprises cells of the present invention.

The method for treating and/or preventing a disease may comprise the step of administering an agent which inhibits the secretion or activity of the secreted factor or antibody to a subject to which the cells according to the present invention have been administered.

The method for treating and/or preventing a disease may comprise the step of administering an agent which increases the secretion or activity of the secreted factor or antibody to a subject to which the cells according to the present invention have been administered.

A method for treating a disease relates to the therapeutic use of the cells of the present invention. In this respect, the cells may be administered to a subject having an existing disease or condition in order to lessen, reduce or improve at least one symptom associated with the disease and/or to slow down, reduce or block the progression of the disease.

The method for preventing a disease relates to the prophylactic use of the cells of the present invention. In this respect, the cells may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

The method may involve the steps of:

(i) isolating a cell-containing sample;

(ii) introducing the nucleic acid construct according to the present invention, a first and second nucleic acid sequence as defined herein, a vector according to the present invention or a first and second vector as defined herein to the cell; and

(iii) administering the cells from (ii) to a subject.

The nucleic acid construct, vector(s) or nucleic acids may be introduced by transduction or transfection. The cell may be autologous or allogenic.

The methods provided by the present invention for treating a disease may involve monitoring the progression of the disease and/or any toxic activity.

The methods provided by the present invention for treating a disease may involve monitoring the progression of the disease and monitoring any toxic activity and adjusting the dose of the agent administered to the subject to provide acceptable levels of disease progression and toxic activity.

“Monitoring the progression of the disease” means to assess the symptoms associated with the disease over time to determine if they are reducing/improving or increasing/worsening.

“Toxic activity” relates to adverse effects caused by the cells of the invention following their administration to a subject. Toxic activities may include, for example, immunological toxicity, biliary toxicity and respiratory distress syndrome.

As such the dose of the agent administered to a subject, or the frequency of administration, may be altered in order to provide an acceptable level of both disease progression and toxic activity. The specific level of disease progression and toxic activities determined to be ‘acceptable’ will vary according to the specific circumstances and should be assessed on such a basis.

The agent may be administered in the form of a pharmaceutical composition. The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

The present invention provides a cell or pharmaceutical composition according to the present invention, a nucleic acid construct according to the present invention for use in treating and/or preventing a disease.

The present invention also relates to the use of a cell according to the present invention for the manufacture of a medicament for the treatment and/or prevention of a disease.

The disease to be treated and/or prevented by the method of the present invention may be cancer.

The cancer may be a cancer such as neuroblastoma, multiple myeloma, prostate cancer, bladder cancer, breast cancer, colon cancer, endometrial cancer, kidney cancer (renal cell), leukaemia, lung cancer, melanoma, non-Hodgkin lymphoma, pancreatic cancer, and thyroid cancer. Suitably, the cancer may be neuroblastoma. Suitably, the cancer may be multiple myeloma. Suitably, the cancer may be prostate cancer.

The cell of the present invention may be capable of killing target cells, such as cancer cells. The target cell may be recognisable by expression of a TAA, for example the expression of a TAA provided above in Table 1. The cancer may be a cancer listed in Table 1.

The administration of a cell or pharmaceutical composition according to the present invention, can be accomplished using any of a variety of routes that make the active ingredient bioavailable. For example, the active ingredient can be administered by oral and parenteral routes, intranasally, intraperitoneally, intravenously, subcutaneously, transcutaneously or intramuscularly.

### Method of Making a Cell

The cells of the present invention may be generated by introducing DNA or RNA coding for the secreted factor or antibody as defined herein, by one of many means including transduction with a viral vector, transfection with DNA or RNA.

The cell of the invention may be made by:

introducing to a cell (e.g. by transduction or transfection) the polynucleotide according to the present invention, the nucleic acid construct or vector according to the present invention, a first and second nucleic acid sequence as defined herein, a vector or a first and second vector as defined herein.

The cell may be transduced or transfected in vitro or ex vivo. Suitably, the cell may be from a sample isolated from a subject.

### Method of Rendering a Cell Less Susceptible to TGFβ

The present invention also provides a method of rendering a cell less susceptible to TGFβ.

The method may comprise introducing to a cell (e.g. by transduction or transfection): the polynucleotide according to the present invention, the nucleic acid construct or vector according to the present invention, a first and second nucleic acid sequence as defined herein, a vector or a first and second vector as defined herein.

The method of rendering a cell less susceptible to TGFβ signalling may comprise maintaining the cell under conditions which allow the expression of the secreted factor or antibody.

The present invention further relates to the use of secreted factor which binds TβR to render an immune effector cell less susceptible to TGFβ.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of’ as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of’.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### EXAMPLES

### Example 1—Generation and Characterisation of Anti-TGFβRII Single Domain Antibodies (dAb)

A library of single domain antibodies was generated by immunising llamas with TGFβRII-Llama Fc, generating a phage display library, panning with TGFβRII-human Fc and eluting either with TGFβ (competitive elution) or trypsin.

Binding to TGFβRII was investigated using a titration ELISA. Briefly, dAbs were expressed and His-purified and applied to plates which had previously been coated with antigen (TGFbRII-Fc) at 1 μg/ml. Plates coated with CD19-Fc were used as a control. Titrations were performed with various concentrations of dAb, starting at 10 μg/ml and decreasing two-fold (so 5, 2.5, 1.25 μg/ml etc).

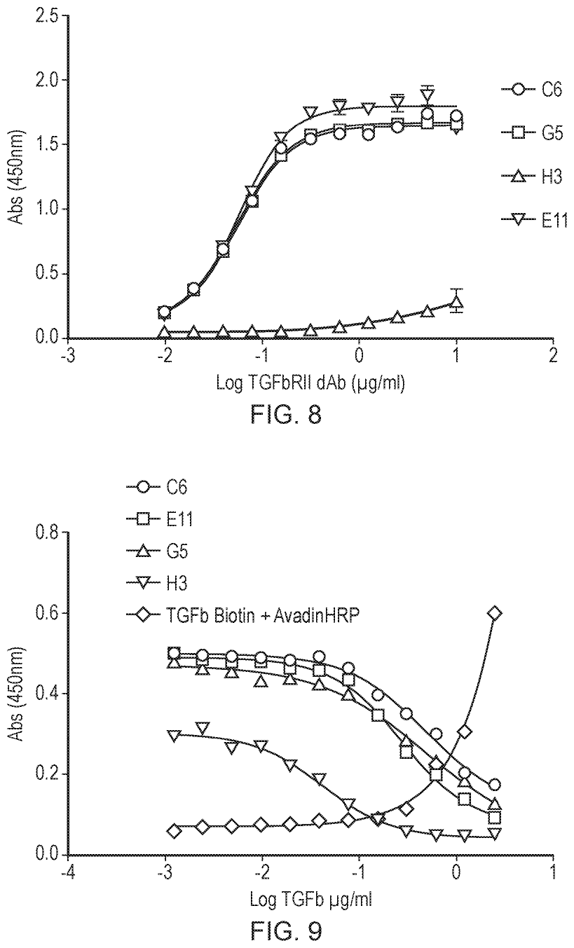

After washing, binding of the dAb was detected using aC-Myc-HRP. The results are shown in FIG. 8.

Four of the dAbs (clones C6, G5, H3 and E11) showed binding to TGFbRII with C6, G5 and E11 showing similar binding curves. Clone H3 shows some binding at the highest antigen concentration.

The binding kinetics of these four dAbs for TGFβRII was then investigated using Biacore affinity analysis and the results are shown in Table 3 below.

TABLE 3

| | 1:1 binding | | |
|---|---|---|---|
| Clone | ka (1/Ms) | kd (1/s) | KD (M) |
| C6 | 1.26e+7 | 4.61e−3 | 3.66e−10 |
| E11 | 9.46e+6 | 9.21e−3 | 9.73e−10 |
| G5 | 8.79e+6 | 4.46e−3 | 5.08e−10 |
| H3 | 1.77e+6 | 1.19e−1 | 6.72e−8 |

The three related dAb binders (C6, E11 and G5) have similar affinities, all of which are in the sub-nanomolar range. The fourth dAb (H3) has a very fast off rate.

Next, it was investigated whether TGFβ binding to TGFβRII can be competed by dAb blockade of TGFbRII, using a competition ELISA. Plates were coated with TGFβRII-Fc (1 ug/ml) as described above and the dAbs were added at 0.25 ug/ml. The capacity of soluble TGFβ to bind plate-bound TGFbRII was then tested by adding TGFβ at 2.5 ug/ml with 2 fold dilutions. TGFβ-Biotin followed by Avadin HRP was used as a positive control and the results are shown in FIG. 9. All four dAbs, including H3, competed with TGFβ for binding to TGFbRII.

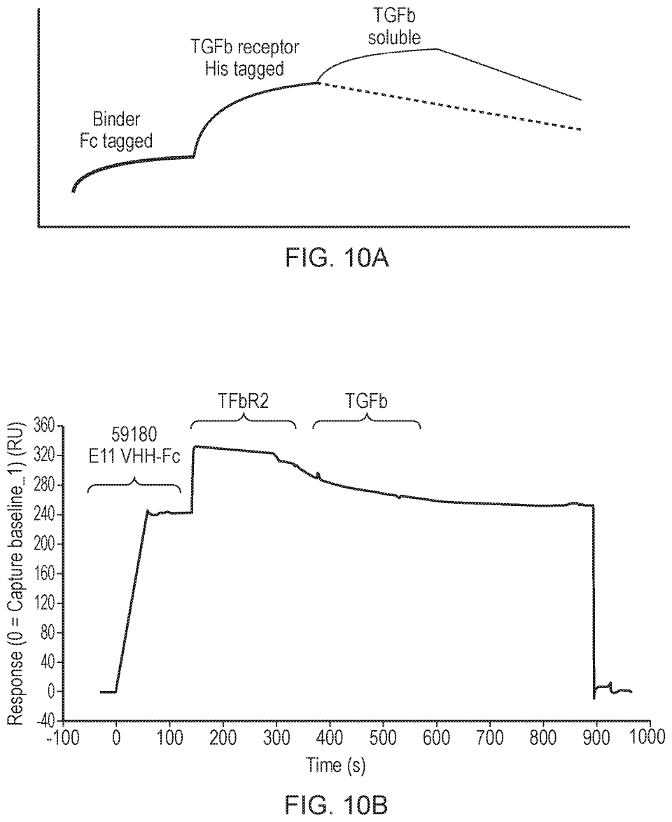

A biacore competition assay was also conducted which involved applying dAb-Fc to the chip surface, binding His-tagged TGFbRII to the dAb and then either adding soluble TGFβ (300 nM) or not. FIG. 10A is a schematic diagram illustrating the results which would be expected if soluble TGFb out-competed the dAb binder for binding TGFβRII. Incremental response units (RU) of the sensograms are shown. The first RU increase represents anti-TGFbRII VHH-Fc binding to the chip surface. The second RU increase represents soluble TGFbRII binding to the captured antibody. The third RU increase represents the binding of TGFbeta to the captured receptor. Sensograms for the TGFbeta 0 and 300 nM injections are superimposed (blue and yellow, respectively) to facilitate identification of TGFbeta 1 binding event. As a representative experiment, the results for clone E11 are shown in FIG. 10B. The addition of TGFβ has no effect—there is no detectable third RU increase—indicating that E11-Fc outcompetes TGFb for binding to TGFbRII.

In order to investigate whether the dAbs bind to TGFβRII when expressed by cells, PBMCs were either left untransfected or transfected with a plasmid to co-express a) an RQR8 marker gene and b) a second generation GD2 CAR (HUK666-CD28z); or a) an RQR8 marker gene, b) a GD2 CAR and c) a dominant negative version of TGFbRII. The plasmids used were as follows:

SFG.mR.RQR8-2A-aGD2_huK666_CD8STK-TyrpTM-CD28z

SFG.mR.RQR8-2A-aGD2_huK666_CD8STK-TyrpTM-CD28z-2A-dnTGFbetaRII

The cells were then incubated with each dAb-Fc conjugate and binding was analysed using flow cytometry and a murine Fc (APC).

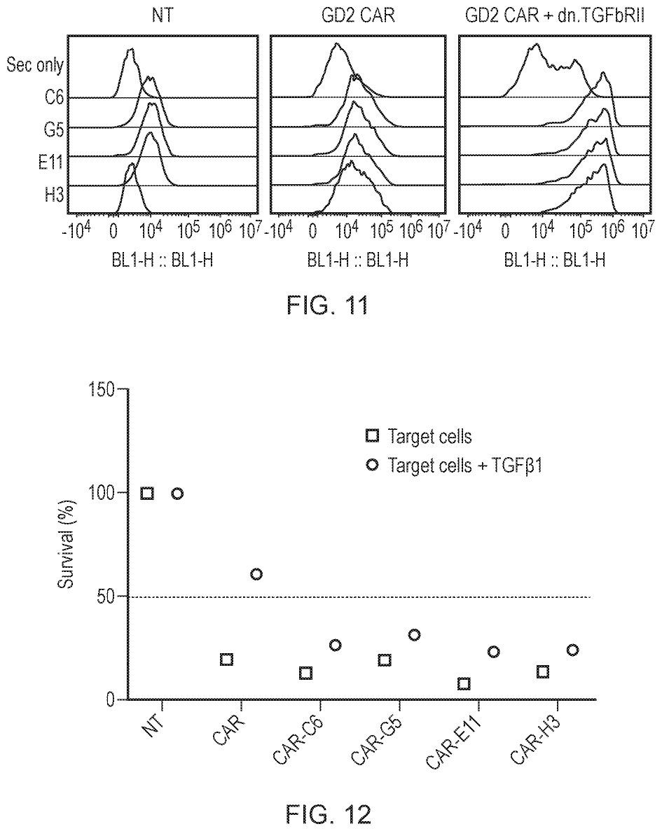

The results are shown in FIG. 11 show that all four anti-TGFbRII dAbs bind to the receptor when expressed on cells expressing dnTGFβRII as part of a CAR construct and not to cells that express CAR only or non-transduced cells.

### Example 2—Generation and CAR-Expressing Cells Secreting the Anti-TGFβRII dAbs

T-cells were created co-expressing a CAR and a secreted anti-TGFbRII dAb.

T cells lines (K562, Jurkat, SupT1 and 293 T-cells) were transfected with a plasmid expressing the CAR and marker gene (as described in Example 1) or co-transfected with this plasmid and a plasmid expressing a dAb-Fc construct, one of C6, G5, E11 and H3. After 3 days, expression of dAb-Fc in the supernatant was analysed by ELISA using TGFbRII-hFc coated plates. Expression of dAb-Fc was detected using murine anti-Fc-HRP. All four cell lines were shown to successfully secrete all four dAbs (data not shown).

### Example 3—the Effect of Anti-TGFβRII dAb Secretion on CAR-Mediated Target Cell Killing

TGFβ has immunosuppressive effects on T cells. Blockade of TGFβRII aims to abrogate the effect of tumour secreted TGFβ by competitively inhibiting receptor binding and thus activation on T cells. In order to investigate this effect, CAR-expressing T cells were used in cytotoxicity assays in the presence or absence of one of the four anti-TGFbRII dAbs. The cytotoxicity assay was set up at a 1:8 effector:target (E:T) cell ratio using SupT1 expressing the antigen as target cells in presence or absence of recombinant human TGFβ1 (10 ng/ml final concentration) in 96-well plates. Non-transduced T cells were used in co-cultures with targets as a negative control. The anti TGFbRII dAb were produced in CHO cells and purified, and then added to the co-culture at 100 ng/ml. CAR-mediated cytotoxicity was assessed by flow cytometry after 5 days. T cells were identified from target cells by anti CD3 antibody, and a viability dye (7AAD) was used to separate live cells from dead cells using an iQue Screener PLUS flow cytometer. Counting beads were added to each sample as quality control. The number of live target cells remaining in the cultures were enumerated and normalized and the percentage cytotoxicity relative to control cultures calculated.

As expected, was found that the addition of TGFβ1 inhibited target cell killing by the T-cells expressing the CAR alone, with more than 50% of target cells surviving at the 5 day point. However, T cells expressing the same CAR which also secreted one of the four anti-TGFbRII dAbs was found to show resistance to such inhibition (FIG. 12).

### Example 4—Proliferation Assay

The transduced T cell population is labelled with the dye Cell Trace Violet (CTV), a fluorescent dye which is hydrolysed and retained within the cell. It is excited by the 405 nm (violet) laser and fluorescence can be detected in the pacific blue channel. The T-cells are resuspended at 2×10⁶ cells per ml in PBS, and 1 ul/ml of 5 mM CTV is added. The T-cells are incubated with the CTV for 20 minutes at 37° C. Subsequently, the cells are quenched by adding 5V of complete media. After a 5 minutes incubation, the T-cells are washed and resuspended in 2 ml of complete media. An additional 10 minute incubation at room temperature allows the occurrence of acetate hydrolysis and retention of the dye.

Labelled T-cells are co-cultured with GD2 expressing target cells for four or seven days. n order to investigate the function of the aTGFBeta dAb modules in the vector, target and effector cells are incubated in the presence or absence of 10 ng/ml TGF Beta.

The assay is carried out in a 96-well plate in 0.2 ml total volume using 5×10⁴ transduced T-cells per well and varying numbers of target cells (ratios—1:2 and 1:8). At day four or day 7 time points, the T-cells are analysed by flow cytometry to measure the dilution of the CTV which occurs as the T-cells divide. The number of T-cells present at the end of the co-culture is calculated and expressed as a fold of proliferation compared to the input number of T cells.

### Example 5—Cytokine Secretion

Cytokine secretion is investigated following co-culture of the CAR-expressing T cells secreting each of the four anti-TGFbRII dAbs with target cells as described in Example 3.

Secretion of IL2 and IFN^{y} is assayed using ELISA assay kits according to the manufacturer's instructions. Briefly, a double antibody sandwich detection ELISA is performed by coating anti-IL2 or ant-IFN^{y} primary antibody onto a Nunc 96 well plate ( 1/1000 dilution and 50 ul/well) or 1h. The plate is washed three times with 100 ul of PBS 0.05% tween, blocked with 100 ul PBS BSA (2%) for 1h and washed again (100 ul of PBS 0.05% tween). The assay supernatant is added to the plate along with an appropriate titration standard and incubated for 1h before washing and detection using a secondary HRP conjugated anti-IL2 or IFN^{y} antibody ( 1/1000 and 50 ul/well). After a final wash the ELISA is developed with OPD and stopped with 1M NaOH (50 ul each). The assays are read on a Varioskan lux plate reader at 450 nm.

### Materials and Methods

### Soluble Antibody Expression and Purification

Anti-TGFbRII antibodies in soluble VHH format were expressed by transient expression on TG1 E. coli strain. Supernatant from IPTG induced TG1 E. coli was purified using metal affinity chromatography. A HisTrap 1 ml column was equilibrated with 5 column volumes of running buffer (300 mM NaCl, 50 mM NaPO4 pH 7.4). Supernatant was applied to the column using Akta™ Pure system at a flow rate of 1 mL/min. Following application of supernatant, the column was washed with 20 column volumes of running buffer. Sample was then eluted from the column with 3 ml of elution buffer (300 mM NaCl, 50 mM NaPO4, 300 mM imidazole, pH 7.4) at 1 mL/min and directly loaded onto 2 HiTrap 5 ml desalting columns, previously equilibrated in PBS, and collected on a 96-well plate using a fraction collector unit. Purity of antibody product was determined via SDS-PAGE.

Anti-TGFbRII antibodies in murine IgG2a Fc format were expressed by transient expression on ExpiCHO cell lines by co-transfection of the relevant plasmid construct.

Supernatant from transfected CHO cells was purified using protein A affinity chromatography. A HiTrap MabSelect SuRE 1 ml column was equilibrated with 5 column volumes of PBS pH 7.4. Supernatant was applied to the column using Akta™ Pure system at a flow rate of 1 mL/min. Following application of supernatant, the column was washed with 20 column volumes of PBS. Sample was then eluted from the column with 3 ml of IgG elution buffer (Pierce—21004) at 1 mL/min and directly loaded onto 2 HiTrap 5 ml desalting columns, previously equilibrated in PBS, and collected on a 96-well plate using a fraction collector unit. Purity of antibody product was determined via SDS-PAGE.

### Affinity Measurement

Recombinant TGFbRII-Fc protein (R&D systems) was immobilised on individual flow cells on a Series S CM5 sensor chip (GE Healthcare) previously functionalised with anti-human capture kit, to a density of 150-200 RU using a Biacore 8K instrument. HBS-P⁺ buffer was used as running buffer is all experimental conditions. Recombinant purified VHH antibodies at known concentrations were used as the ‘analyte’ and injected over the respective flow cells with 150 s contact time and 600s dissociation at 30 pl/minute of flow rate with a constant temperature of 25° C. In each experiment, flow cell 1 was unmodified and used for reference subtraction. A ‘0 concentration’ sensorgram of buffer alone was used as a double reference subtraction to factor for drift. Data were fit to a 1:1 Langmuir binding model using local Rmax.

### Biacore Competition Assay

To assess antibodies' ability to compete for soluble TGFbeta1 binding to TGFbRII, purified anti-TGFbRII VHH-Fc antibodies were captured on a Series S CM5 sensor chip (GE Healthcare) to a density of 200-250 RU on individual flow cells, using a Biacore 8K instrument. Antibody contact time was maintained for 60s at 10 ul/min in HBS-P⁺ buffer. Soluble TGFbRII recombinant protein (R&D biosystem) was injected for 150s at 30 ul/min over the relevant flow cells at a concentration of 1 uM. Finally, recombinant TGFbeta1 protein (Acro biosystem) was injected for 150s at 30 ul/min over the relevant flow cells, with 300s dissociation time, at a concentration of 0 and 300 nM.

All publications mentioned in the above specification are herein incorporated by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1-5. (canceled)

6. A domain antibody (dAb) which binds transforming growth factor beta receptor type II (TβRII) and comprises one of the following sets of complementarity determining regions (CDRs):
(SEQ ID No. 1)
a) CDR1-GIDLSTKS;
(SEQ ID No. 2)
CDR2-HSSSGGTT;
and
(SEQ ID No. 3)
CDR3-TAPVGY;
(SEQ ID No. 4)
b) CDR1-GIILSSKA;
(SEQ ID No. 5)
CDR2-HSSVSGTT;
and
(SEQ ID No. 6)
CDR3-TAPVGH;
(SEQ ID No. 7)
c) CDR1-GITFSTKP;
(SEQ ID No. 8)
CDR2-HISSDGTT
and
(SEQ ID No. 9)
CDR3-TAPVGY;
or
(SEQ ID No. 10)
d) CDR1-GRSFSIYG;
(SEQ ID No. 11)
CDR2-ITGSGLTT;
and
(SEQ ID No. 12)
CDR3-AATRAYSGYYGENPTRYAY.

7. A dAb according to claim 6, which comprises one of the sequences shown as SEQ ID No. 13, 14, 15 or 16.

8. A nucleic acid sequence encoding a dAb according to claim 6.

9. A nucleic acid construct which comprises: (i) a first polynucleotide which encodes an antibody as defined in claim 6; and (ii) a second polynucleotide which encodes a chimeric antigen receptor (CAR) or a transgenic T-cell receptor (TCR).

10. A nucleic acid construct according to claim 9, wherein the first and second polynucleotides are separated by a co-expression site.

11. A kit of polynucleotides comprising:
(i) a first polynucleotide which encodes a dAb according to claim 6; and
(ii) a second polynucleotide which encodes a chimeric antigen receptor (CAR) or a transgenic (TCR).

12. A vector which comprises a nucleic acid sequence according to claim 8.

13. A kit of vectors which comprises:
(i) a first vector which comprises a polynucleotide which encodes a dAb according to claim 6; and
(ii) a second vector which comprises a polynucleotide which encodes a chimeric antigen receptor (CAR) or a transgenic T-cell receptor (TCR).

14. A pharmaceutical composition which comprises a dAb according to claim 6.

15. (canceled)

16. A method for treating a disease, which comprises the step of administering a pharmaceutical composition according to claim 14 to a subject in need thereof.

17. (canceled)

18. The method according to claim 16, wherein the disease is cancer.

19. A method for making a cell according to claim 20, which comprises the step of introducing: a first polynucleotide encoding a domain antibody (dAb) which binds transforming growth factor beta receptor type II (TβRII) and comprises one of the following sets of complementarity determining regions (CDRs)
a) CDR1—GIDLSTKS (SEQ ID No. 1);
CDR2—HSSSGGTT (SEQ ID No. 2); and
CDR3—TAPVGY (SEQ ID No. 3);
b) CDR1—GIILSSKA (SEQ ID No. 4);
CDR2—HSSVSGTT (SEQ ID No. 5); and
CDR3—TAPVGH (SEQ ID No. 6);
c) CDR1—GITFSTKP (SEQ ID No. 7);
CDR2—HISSDGTT (SEQ ID No. 8); and
CDR3—TAPVGY (SEQ ID No. 9); or
d) CDR1—GRSFSIYG (SEQ ID No. 10);
CDR2—ITGSGLTT (SEQ ID No. 11); and
CDR3—AATRAYSGYYGENPTRYAY (SEQ ID No. 12); and a second polynucleotide which encodes a chimeric antigen receptor (CAR) or a transgenic T-cell receptor (TCR), into a cell ex vivo.

20. A cell which comprises a chimeric antigen receptor (CAR) or transgenic T cell receptor (TCR) and secretes a dAb according to claim 1.

21. A pharmaceutical composition which comprises a cell according to claim 20.

22. A vector which comprises a nucleic acid construct according to claim 9.
